(19) United States
(12) Patent Application Publication
(10) Pub. No.: US 2010/0279280 A1
(43) Pub. Date: Nov. 4, 2010

(54) FUNCTIONAL POLYMORPHISMS OF THE INTERLEUKIN-1 LOCUS AFFECTING TRANSCRIPTION AND SUSCEPTIBILITY TO INFLAMMATORY AND INFECTIOUS DISEASES

(76) Inventors: David Wyllie, Headington (GB); Gordon Duff, Sheffield (GB); Nazneen Aziz, Lexington, MA (US); Chung-Ming Hsieh, West Roxbury, MA (US); Kenneth Kornman, Newton, MA (US)

Correspondence Address: Ivor R. Elrifi;Mintz, Levin, Cohn, Ferris, Glovsky and Popeo, P.C, One Financial Center, Boston, MA, 02111, US

(21) Appl. No.: 12/048,077

(22) Filed: Mar. 13, 2008

Related U.S. Application Data
(63) Continuation of application No. 10/300,011, filed on Nov. 19, 2002, now abandoned.
(60) Provisional application No. 60/386,020, filed on Jun. 5, 2002.
(60) Provisional application No. 60/331,681, filed on Nov. 19, 2001.

(51) Int. Cl. C12Q 1/68; G01N 33/564; G01N 33/50

(52) U.S. Cl. 435/6; 436/506; 436/94

(57) ABSTRACT

The invention provides methods and reagents for detecting a polymorphism associated with in an upstream region of the interleukin-1 beta (IL-B) gene (IL-1B (−3737)) that affects transcription of the gene and susceptibility to inflammatory and infectious diseases such as periodontal disease and Alzheimer's disease.

### 1. BACKGROUND OF THE INVENTION

The IL-1 gene cluster is on the long arm of chromosome 2 (2q13) and contains at least the genes for IL-1α (IL-1A), IL-1β (IL-1B), and the IL-1 receptor antagonist (IL-1RN), within a region of 430 Kb (Nicklin, et al. (1994) Genomics, 19: 382-4). The agonist molecules, IL-1α and IL-1β, have potent pro-inflammatory activity and are at the head of many inflammatory cascades. Their actions, often via the induction of other cytokines such as IL-6 and IL-8, lead to activation and recruitment of leukocytes into damaged tissue, local production of vasoactive agents, fever response in the brain and hepatic acute phase response. All three IL-1 molecules bind to type I and to type II IL-1 receptors, but only the type I receptor transduces a signal to the interior of the cell. In contrast, the type II receptor is shed from the cell membrane and acts as a decoy receptor. The receptor antagonist and the type II receptor, therefore, are both anti-inflammatory in their actions.

Inappropriate production of IL-1 plays a central role in the pathology of many autoimmune and inflammatory diseases, including rheumatoid arthritis, inflammatory bowel disorder, psoriasis, and the like. In addition, there are stable inter-individual differences in the rates of production of IL-1, and some of this variation may be accounted for by genetic differences at IL-1 gene loci. Thus, the IL-1 genes are reasonable candidates for determining part of the genetic susceptibility to inflammatory diseases, most of which have a multifactorial etiology with a polygenic component.

Certain alleles from the IL-1 gene cluster are known to be associated with particular disease states. For example, IL-1RN (VNTR) allele 2 has been shown to be associated with osteoporosis (U.S. Pat. No. 5,698,399), nephropathy in diabetes mellitus (Blakemore, et al. (1996) Hum. Genet. 97(3): 369-74), alopecia areata (Cork, et al., (1995) J. Invest. Dermatol. 104(5 Supp.): 15S-16S; Cork et al. (1996) Dermatol Clin 14: 671-8), Graves disease (Blakemore, et al. (1995) J. Clin. Endocrinol. 80(1): 111-5), systemic lupus erythematosus (Blakemore, et al. (1994) Arthritis Rheum. 37: 1380-85), lichen sclerosis (Clay, et al. (1994) Hum. Genet. 94: 407-10), and ulcerative colitis (Mansfield, et al. (1994) Gastoenterol. 106(3): 637-42)).

In addition, the IL-1A allele 2 from marker −889 and IL-1B (TaqI) allele 2 from marker +3954 have been found to be associated with periodontal disease (U.S. Pat. No. 5,686,246; Kornman and diGiovine (1998) Ann Periodont 3: 327-38; Hart and Kornman (1997) Periodontol 2000 14: 202-15; Newman (1997) Compend Contin Educ Dent 18: 881-4; Kornman et al. (1997) J. Clin Periodontol 24: 72-77). The IL-1A allele 2 from marker −889 has also been found to be associated with juvenile chronic arthritis, particularly chronic iridocyclitis (McDowell, et al. (1995) Arthritis Rheum. 38: 221-28). The IL-1B (TaqI) allele 2 from marker +3954 of IL-1B has also been found to be associated with psoriasis and insulin dependent diabetes in DR3/4 patients (diGiovine, et al. (1995) Cytokine 7: 606; Pociot, et al. (1992) Eur J. Clin. Invest. 22: 396-402). Additionally, the IL-1RN (VNTR) allele 1 has been found to be associated with diabetic retinopathy (see U.S. Ser. No. 09/037472, and PCT/GB97/02790). Furthermore allele 2 of IL-1RN (VNTR) has been found to be associated with ulcerative colitis in Caucasian populations from North America and Europe (Mansfield, J. et al., (1994) Gastroenterology 106: 637-42). Interestingly, this association is particularly strong within populations of ethnically related Ashkenazi Jews (PCT WO97/25445). In addition, extensive methods and compositions for the detection and association of IL-1 polymorphisms with inflammatory disease have been described in U.S. Pat. Nos. 5,685,246, 5,698,399, 6,140,047, 6,251,598, and 6,268,142, the contents of which are incorporated herein by reference. In addition, transgenic models for IL-1 locus based inflammatory disease are described in U.S. Pat. No. 6,437,216, the contents of which are incorporated herein by reference.

Traditional methods for the screening of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes such as, for example, vascular disease. With the development of simple and inexpensive genetic screening methodology, it is now possible to identify polymorphisms that indicate a propensity to develop disease, even when the disease is of polygenic origin. The number of diseases that can be screened by molecular biological methods continues to grow with increased understanding of the genetic basis of multifactorial disorders.

Genetic screening (also called genotyping or molecular screening), can be broadly defined as testing to determine if a patient has mutations (alleles or polymorphisms) that either cause a disease state or arc “linked” to the mutation causing a disease state. Linkage refers to the phenomenon where DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

The statistical correlation between an inflammatory disorder and an IL-1 polymorphism does not necessarily indicate that the polymorphism directly causes the disorder. Rather the correlated polymorphism may be a benign allelic variant which is linked to (i.e. in linkage disequilibrium with) a disorder-causing mutation which has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype. Thus, the determination of an individual's likelihood for developing a particular disease of condition can be made by characterizing one or more disease-associated polymorphic alleles (or even one or more disease-associated haplotypes) without necessarily determining or characterizing the causative genetic variation.

Nevertheless, although the detection of one or more linked alleles in an IL-1 haplotype that have been statistically associated with a propensity to develop a particular inflammatory disease or condition provides a useful diagnostic method for predicting and treating inflammatory disease, ultimately the most reliable polymorphic indicators will those alleles which are most strongly associated with an underlying element of the etiology of the disease (i.e. causative mutations or “functional alleles”).

For example, many studies throughout the world have shown that three chemicals in the tissues are consistently associated with more severe disease or actively progressing disease. Those chemicals are interleukin-1 (IL-1), prostaglandin-E2 (PGE₂) and the enzymes that destroy collagen and bone matrix metalloproteinases (MMPs) (see Offenbacher, S. (1996), Ann. Periodontol. 1:821; Page, R. C. and Kornman, K. S. (1997), Periodontology 2000 14:112). These chemicals are important mediators of the inflammatory response and appear to play a central role in bone loss. IL-1 is a primary regulator of both PGE₂ and matrix metalloproteinases. Recent studies (see Assuma, R. et al. (1998), J. Immunol. 160:403) showed that specific blocking of IL-1 and TNFα in the gingival tissues, without any plaque control measures, blocked a substantial part of the bone loss in a monkey model of periodontal disease. There are many reports on IL-1 levels in tissue and gingival crevice fluid (GCF) or IL-1 production from cells and association with bone loss and more advanced or progressive periodontitis (see e.g. Gemmell, E. and Seymour, G. J. (1998), J. Dent. Res. 77:16; Ishihara, Y. et al. (1997), J. Periodontal Res. 32:524; McGee, J. M. et al. (1998), J. Periodontol. 69:865; Okada, H. and Murakami, S. (1998), Crit. Rev. Oral Biol. Med. 9:248; Roberts, F. A. et al. (1997), Oral Microbiol. Immunol. 12:336; Salvi, G. E. et al. (1998), J. Periodontal Res. 33:212; Stashenko, P. et al. (1991), J. Clin. Periodontol. 18:548; Yavuzyilmaz, E. et al. (1995), Aust. Dent. J. 40(1):46). For example, recent studies (see Cavanaugh, P. F. et al. (1998), J. Periodont. Res. 33:75), looking at the severity of bone loss compared to gingival crevicular fluid levels of IL-1 indicate that higher levels of IL-1 in the crevicular fluid are associated with relatively more bone loss.

Recently, the critical role of IL-1 in bone destruction was shown in a mouse model, (Lorenzo, J. A. et al. (1998), Endocrinology 139(6):3022). When mice with an intact IL-1 system were ovariectomized to stimulate estrogen depletion during menopause, the animals lost substantial bone density. When mice were created with a blockage in the IL-1 system, the estrogen depletion resulted in no bone loss. This suggests that, at least in mice, IL-1 is essential for bone loss after estrogen depletion. IL-1 was found to be an essential part of periodontitis in other studies (see Assuma, R. et al. (1998), J. Immunol. 160:403). The investigators produced periodontitis in monkeys. One group of monkeys was treated with chemicals that specifically block IL-1 and a similar chemical, TNFα. The animals with blocked IL-1 and TNFα developed much less bone loss, in spite of having a heavy bacterial challenge.

It has been known for several years that some people produce higher levels of IL-1 than other people. The high producers on one day will also be high producers if examined again at a later date, and high production of IL-1 tends to run in families. It is not known that there are specific IL-1 gene variations that cause high production of IL-1 when that individual is exposed to a bacterial challenge. Approximately 30% of Caucasians have these genetic factors.

In some studies, peripheral white blood cells (see Mark, L. L. et al. (2000), J. Periodontal Res. 35(3):172; diGiovine, F. S. et al. (1995), Cytokine 7:606; Pociot, F. et al. (1992), Eur. J. Clin. Invest. 22:396; Galbraith, G. M. et al. (1997), J. Periodontol. 68:832), incubated in the laboratory with bacterial products from gram-negative bacteria, produced significantly more IL-1β if the white blood cells have come from a person who has a specific variation in the IL-1 genes (“genotype positives”). Perhaps most importantly, however, the levels of IL-1 are higher in the periodontal tissues of genotype positives. In recent studies the IL-1α and IL-1β levels were significantly higher in the gingival crevicular fluid of genotype positive patients than those of genotype negative patients (see Engebretson, S. P. et al. (1999), J. Periodontol. 70(6):567; Shirodaria, S. et al. (2000), J. Dent. Res. 79(11):1864). In fact, in one of the studies (Engebretson, S. P. et al. (1999), J. Periodontol. 70(6):567), the greatest difference between genotype positives and genotype negatives was found in sites with minimal pocket depth (<4 mm).

In addition, bleeding on probing may be considered as a clinical indicator of the inflammatory response. Lang and co-workers (see Lang, N. P. et al. (2000), J. Periodontal. Res. 35(2):102), evaluated over 320 randomly selected patients in a clinical recall program. Out of 139 non-smokers, genotype positive patients were significantly more likely than genotype negatives to have an increase in number of bleeding sites during four maintenance visits.

In summary, patients who are positive for the IL-1 genotype tend to have: a) increased IL-1 levels produced by their white blood cells, 2) increased IL-1 in the gingival crevicular fluid, and 3) increased bleeding on probing.

Diagnostic tools are used to identify some aspect of a disease that is already present. Examples of diagnostic test include not only radiographs but biochemical markers of active bone loss. The evaluation of value for a specific diagnostic is based on the assessment of how well the diagnostic detects the disease change when it is actually present and how well the test avoids being “positive” when there is actually no disease.

Prognostics in medicine and dentistry are intended to forecast risk for future aspects of disease. Since there are no facts about the future, prognostics involve a probability of future events occurring. All patients are familiar with the concept of forecasts. A weather forecast of a 60% chance of rain does not guarantee that it will rain, but given that forecast, most people would select different clothing for the day. Similarly, high cholesterol does not guarantee that one will have a heart attack in the future, but it more than doubles the chance of an acute coronary event before a certain age.

People who are positive for the IL-1 genotype are more likely to have generalized severe periodontitis (see e.g. Gore, E. A. et al. (1998), J. Clin. Periodontol. 25:781, Kornman, K. S. and diGiovine, F. S. (1998), Ann. Periodontol. 3:327; Kornman, K. S. et al. (1997), J. Clin. Periodontol. 24:72; McDevitt, M. J. et al. (2000), J. Periodontal 71:156. In a recent study, McDevitt, M. J. et al. (2000), J. Periodontal 71:156, 90) subjects with no or minimal smoking history were examined for periodontal disease and IL-1 genotypes. Multivariate regression models demonstrated that a patient's age, former smoking history and IL-1 genotype were significantly associated with the severity of periodontal bone loss in adults. For non-smokers or former light smokers (<5 pk-yr), IL-1 genotype positives were more than three times more likely to have moderate to severe periodontal disease than patients who were IL-1 genotype negative.

In a study on a periodontal maintenance patient population (see McGuire and Nunn, McGuire, M. K. et al. (1999), J. Periodontol. 70(1):49), examined patients who had been followed for 5-14 years after periodontal therapy. They attempted to determine what, if any, factors predicted tooth loss in patients during the periodontal maintenance phase. They found that only two predictors: IL-1 genotype and heavy smoking were significantly related to later tooth loss. IL-1 positive genotype were 2.7 times more likely to have tooth loss than genotype negatives, and heavy smokers were 2.9 times more likely to have tooth loss than genotype positives. Patients who were both genotype positive and also heavy smokers were 7.7 times more likely to have tooth loss than non-smokers who were genotype negative. The clinical parameters traditionally used to assign prognosis were found to be valuable only in IL-1 genotype negative patients who were non-smokers.

In another study, predictors of treatment outcomes were evaluated. Furthermore, another study (see DeSanctis, M. and Zuchelli, G. (2000), J. Periodontol. 71:606) indicated that long-term stability of periodontal tissue after guided tissue regeneration (GTR) surgery to regenerate the destroyed periodontal attachment was significantly descreted in genotype positive patients (see DeSanctis, M. and Zuchelli, G. (2000), J. Periodontol. 71:606).

It is important to emphasize that chronic diseases, such as periodontitis, involve complex biological interactions over time. The relationship between IL-1 gene expression and a few single-nucleotide polymorphisms is a particularly critical aspect of that complex biology. Accordingly, a functional polymorphism which results in increased production of IL-1B or IL-1A (or other IL-1 locus gene) is useful in the prediction and diagnosis of periodontal as well as other inflammatory diseases and conditions which have been associated with increased production of IL-1beta or IL-1alpha. For example, increased production of IL-1B has been shown to play a role in the etiology of rheumatoid arthritis, Alzheimer's disease, inflammatory bowel disease, and graft-versus-host disease (see e.g. Dinarello (2000) Chest 118: 503-08 for review). Furthermore, functional polymorphisms associated with decreased expression of an IL-1 locus gene can also play a role in inflammatory disease. For examples, functional polymorphisms that cause a decrease in the expression of IL-1RN (the IL-1 locus receptor antagonist) also can result in elevated interleukin levels and resultant inflammatory disease. Accordingly, it would be useful to identify functional polymorphisms in the IL-1 locus that affect transcription or expression of one or more IL-1 genes.

### 2. SUMMARY OF THE INVENTION

In one aspect, the present invention provides novel methods and kits for determining whether a subject has or is predisposed to developing a disease or condition that is associated with increased production of interleukin, particularly IL-1 beta. In one embodiment, the method comprises determining whether the subject's nucleic acids contains an IL-1B (−3737) polymorphic allele. In a preferred embodiment, the IL-1B (−3737) allele detected is a type 1 allele associated with increased IL-1B expression and associated with inflammatory disease, however detection of the type 2 allele is useful—particularly inasmuch as it confirms absence of the type 1 allele on one or both chromosomes of the test subject.

In a particularly preferred embodiment, the invention provides an isolated nucleic acid which includes about 20 contiguous nucleotides of genomic sequence from the human IL-1B (−3737) polymorphic locus. Preferred nucleic acids include those corresponding to the −3737 IL-1B allele 1 sequence: TCTAGACCAGGGAGGAGAATGGAATGTCCCTTGGACTCTGCA-TGT; as well as those corresponding to the −3737 IL-1B allele 2 sequence: TCTAGACCAGG-GAGGAGAATGGAATGTTCCTTGGACTCTGCATGT.

In another embodiment, the invention provides an isolated nucleic acid which includes about 20 contiguous nucleotides of genomic sequence from the human IL-1B (−1469) polymorphic locus. Preferred nucleic acids include those corresponding to the −1469 IL-1B allele 1 sequence: ACAGAGGCTCACTCCCTTGCATAATGCAGAGCGAGCACGATACC-TGG; as well as those corresponding to the −1469 IL-1B allele 2 sequence: ACAGAGGCTCA-CTCCCTTGTATAATGCAGAGCGAGCACGATACCTGG.

In still another embodiment, the invention provides an isolated nucleic acid which includes about 20 contiguous nucleotides of genomic sequence from the human IL-1B (−999) polymorphic locus. Preferred nucleic acids include those corresponding to the −999 IL-1B allele 1 sequence: GATCGTGCCACTgcACTCCAGCCTGGGCGACAGGGTGAGACTCTGTCTC; as well as those corresponding to the −999 IL-1B allele 2 sequence: GATCGTGCCACTgc-ACTCCAGCCTGGGCGACAGCGTGAGACTCTGTCTC.

In other embodiments, the nucleic acid of the invention include a sequence complementary to any of those described above, as well as allele-specific oligonucleotides such as those with a 3′ end which corresponds to an allelic variant at the −3737, −1469 or −999 IL-1B polymorphic locus. Particularly preferred nucleic acids are probes which contain one of the above described sequences as well as a detectable label.

In another particularly preferred embodiment, the invention provides methods of predicting or diagnosing an increased likelihood of developing an inflammatory disease or condition in a human subject. In this aspect of the invention, the inflammatory diseases is one associated with increased expression of interleukin, particularly IL-1B, and the method requires that a sample of nucleic acid be obtained from the human subjected and analyzed to determine the identity of the −3737 IL-1B allele as a type 1 or a type 2 promoter sequence. The presence of a type 1 IL-1B promoter sequence is diagnostic of an increased likelihood of developing an inflammatory disease. This aspect of the invention is particularly useful for diagnosing an inflammatory disease or condition associated with increased interleukin production, particularly IL-1B production, such as periodontal disease and Alzheimer's disease.

Still other inflammatory diseases and conditions which can be diagnosed or predicted by the method of the invention include The phrase “diseases and conditions associated with IL-1 polymorphisms” refers to a variety of diseases or conditions, the susceptibility to which can be indicated in a subject based on the identification of one or more alleles within the IL-1 complex. Examples include: inflammatory or degenerative disease, including: Systemic Inflammatory Response (SIRS); Alzheimer's Disease (and associated conditions and symptoms including: chronic neuroinflammation, glial activation; increased microglia; neuritic plaque formation; and response to therapy); Amylotropic Lateral Sclerosis (ALS), arthritis (and associated conditions and symptoms including: acute joint inflammation, antigen-induced arthritis, arthritis associated with chronic lymphocytic thyroiditis, collagen-induced arthritis, juvenile chronic arthritis; juvenile rheumatoid arthritis, osteoarthritis, prognosis and streptococcus-induced arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, glomerulonephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyperresponsiveness), Ophthalmopathies (and associated diseases and conditions, including diabetic retinopathy, Graves' Ophthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis; rheumatism including rheumatoid arthritis, rheumatic aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Immunological disorders, including autoimmune diseases, such as alopecia aerata, autoimmune myocarditis, Graves' disease, Graves ophthalmopathy, lichen sclerosis, multiple sclerosis, psoriasis, systemic lupus erythematosus, systemic sclerosis, thyroid diseases (e.g. goiter and struma lymphomatosa (Hashimoto's thyroiditis, lymphadenoid goiter), sleep disorders and chronic fatigue syndrome and obesity (non-diabetic or associated with diabetes). Resistance to infectious diseases, such as Leishmaniasis, Leprosy, Lyme Disease, Lyme Carditis, malaria, cerebral malaria, meningititis, tubulointestitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosomes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries). Susceptibility to neoplasias, including breast cancer associated osteolytic metastasis, cachexia, colorectal cancer, hyperproliferative diseases, Hodgkin's disease, leukemias, lymphomas, metabolic diseases and tumors, metastases, myeolomas, and various cancers (including breast prostate ovarian, colon, lung, etc), anorexia and cachexia. Hormonal regulation including fertility/fecundity, likelihood of a pregnancy, incidence of preterm labor, prenatal and neonatal complications including preterm low birth weight, cerebral palsy, septicemia, hypothyroxinernia, oxygen dependence, cranial abnormality, early onset menopause. A subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

Another aspect of the invention provides methods of determining whether a human subject can be effectively treated with a therapeutic drug by testing a sample of the human subject's nucleic acid and determining the identity of the −3737 IL-1B allele as a type 1 or a type 2 promoter sequence. In preferred embodiments of this aspect of the invention, the presence of a type 1 IL-1B promoter sequence indicates that the human subject can be effectively treated with the therapeutic drug.

In another embodiment, the IL-1B (−3737) type 2 allele is a component of an IL-1 inflammatory haplotype and its presence is indicative of increased Il-1beta expression (e.g. IL-1 (3344146)). In a preferred embodiment of this aspect of the invention, the invention provides methods for diagnosing or predicting an increased likelihood of developing an inflammatory disease or condition associated with increased interleukin production by detecting the presence of an IL-1 haplotype associated with a −3737 IL-1B type 1 allele, wherein the presence of the IL-1 haplotype associated with the −3737 IL-1B type 1 allele is diagnostic of an increased likelihood of developing the inflammatory disease or condition.

An allele comprising an IL-1 inflammatory haplotype can be detected by any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the IL-1 gene loci, either flanking the marker of interest (as required for PCR amplification) or directly overlapping the marker (as in ASO hybridization). In a particularly preferred embodiment, the sample is hybridized with a set of primers, which hybridize 5′ and 3′ in a sense or antisense sequence to the vascular disease associated allele, and is subjected to a PCR amplification.

An allele comprising an IL-1 inflammatory haplotype may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications.

In another aspect, the invention features kits for performing the above-described assays. The kit can include a nucleic acid sample collection means and a means for determining whether a subject carries at least one allele comprising an IL-1 inflammatory haplotype. The kit may also contain a control sample either positive or negative or a standard and/or an algorithmic device for assessing the results and additional reagents and components including: DNA amplification reagents, DNA polymerase, nucleic acid amplification reagents, restrictive enzymes, buffers, a nucleic acid sampling device, DNA purification device, deoxynucleotides, oligonucleotides (e.g. probes and primers) etc.

As described above, the control may be a positive or negative control. Further, the control sample may contain the positive (or negative) products of the allele detection technique employed. For example, where the allele detection technique is PCR amplification, followed by size fractionation, the control sample may comprise DNA fragments of the appropriate size. Likewise, where the allele detection technique involves detection of a mutated protein, the control sample may comprise a sample of mutated protein. However, it is preferred that the control sample comprises the material to be tested. For example, the controls may be a sample of genomic DNA or a cloned portion of the IL-1 gene cluster. Preferably, however, the control sample is a highly purified sample of genomic DNA where the sample to be tested is genomic DNA.

The oligonucleotides present in said kit may be used for amplification of the region of interest or for direct allele specific oligonucleotide (ASO) hybridization to the markers in question. Thus, the oligonucleotides may either flank the marker of interest (as required for PCR amplification) or directly overlap the marker (as in ASO hybridization).

Information obtained using the assays and kits described herein (alone or in conjunction with information on another genetic defect or environmental factor, which contributes to the disease or condition that is associated with an IL-1 inflammatory haplotype) is useful for determining whether a non-symptomatic subject has or is likely to develop the particular disease or condition. In addition, the information can allow a more customized approach to preventing the onset or progression of the disease or condition. For example, this information can enable a clinician to more effectively prescribe a therapy that will address the molecular basis of the disease or condition.

In yet a further aspect, the invention features methods for treating or preventing the development of a disease or condition that is associated with an IL-1 inflammatory haplotype in a subject by administering to the subject an appropriate therapeutic of the invention. In still another aspect, the invention provides in vitro or in vivo assays for screening test compounds to identify therapeutics for treating or preventing the development of a disease or condition that is associated with an IL-1 inflammatory haplotype. In one embodiment, the assay comprises contacting a cell transfected with a causative mutation that is operably linked to an appropriate promoter with a test compound and determining the level of expression of a protein in the cell in the presence and in the absence of the test compound. In a preferred embodiment, the causative mutation results in decreased production of IL-1 receptor antagonist, and increased production of the IL-1 receptor antagonist in the presence of the test compound indicates that the compound is an agonist of IL-1 receptor antagonist activity. In another preferred embodiment, the causative mutation results in increased production of IL-1α or IL-1β, and decreased production of IL-1α or IL-1β in the presence of the test compound indicates that the compound is an antagonist of IL-1α or IL-1β activity. In another embodiment, the invention features transgenic non-human animals and their use in identifying antagonists of IL-1α or IL-1β activity or agonists of IL-1Ra activity.

In another embodiment, the invention provides methods for predicting the likelihood of developing an inflammatory disease or condition associated with altered IL-1B expression in a human subject by detecting, in a sample of nucleic from the human subject an IL-1B, any of the following polymorphisms: IL-1B4 allele1 (TGCATAGGGTC), IL-1B3 allele 1 (TGCATAGGGTC), IL-1B7 allele-1 (TGCATAGGGTC), IL-1B15 allele 1 (TGCATAGGGTC), IL-1B4 allele2 (TGTATAGGGTC), IL-1B3 allele 2 (TACATAGGGTC), IL-1B7 allele-2 (TGCATGGGGTC), and IL-1B15 allele 2 (TGCATAGGGTT). Also included in the invention are nucleic acids for the detection of an IL-1 inflammatory genotype such as isolated nucleotides comprising an IL-1B SNP such as IL-1B4 allele1 (TGCATAGGGTC), IL-1B3 allele 1 (TGCATAGGGTC), IL-1B7 allele-1 (TGCATAGGGTC), IL-1B15 allele 1 (TGCATAGGGTC), IL-1B4 allele2 (TGTATAGGGTC), IL-1B3 allele 2 (TACATAGGGTC), IL-1B7 allele-2 (TGCATGGGGTC), or IL-1B15 allele 2 (TGCATAGGGTT).

In a particularly preferred aspect, the invention provides methods for detecting a functional polymorphism associated with altered IL-1 gene expression by identifying an IL-1 SNP, and functionally assessing the effect of the SNP on IL-1 gene expression or binding of an IL-1 gene transcription factor. By this method, when the SNP is associated with altered IL-1 gene expression or altered binding of an IL-1 gene transcription factor, then the SNP is a functional polymorphism associated with altered IL-1 gene expression and, accordingly, is associated with an altered likelihood of developing an inflammatory disease or condition.

Other embodiments and advantages of the invention are set forth in the following detailed description and claims.

### 3. BRIEF DESCRIPTION OF THE FIGURES

FIG. 1 shows the sequence of the IL-1B gene, including the upstream promoter region—the −3737 allele 1 is in bold and the corresponding detection oligonucleotide is underlined (see GenBank Accession Nos. X04500 and AC04500); the −1469 and −999 polymorphism detection oligonucleotides and respective polymorphic sites are also underlined and bolded.

FIG. 2 shows a variation in IL-1B transcription rate that is associated with an IL-1B genotype.

FIG. 3 shows a schematic representation of the IL-1B proximal promoter and distal enhancer genomic region.

FIG. 4 shows that there is no influence of −31 and −511 polymorphism status upon transcriptional activity of IL1B promoter.

FIG. 5 shows the strategy for cloning of the IL-1B upstream promoter region.

FIG. 6 shows the transcriptional differences between −511 type 1 and type 2 promoters.

FIG. 7 shows the dose/response relationship—type 1 vs. type 2 clones.

FIG. 8 shows the dose and time responsiveness of type 1 and type 2 IL-1B clones.

FIG. 9. shows the binding of NF-kB p50 homodimers to DNA substrate.

FIG. 10 shows the transfection analysis of −3737 (also known as IL-1B4 as per annotation of the SNP discovery results) SNP into RAW cells (murine macrophage cells)

FIG. 11 shows the sequence of the IL-1B constructs tested in the functional polymorphism transfection analyses.

FIG. 12 Shows the results from functional analysis of additional functional SNPs in THP-1 cells.

### 4. DETAILED DESCRIPTION OF THE INVENTION

### 4.1. General

The invention relates to the discovery of a polymorphism in the IL-1B gene which is associated with an altered IL-1 beta production rate. Ascertainment of genotype at this polymorphism provides a useful genetic test for susceptibility to diseases where IL-1 production contributes to pathogenesis—e.g. periodontal disease and other inflammatory diseases, particularly those such as Alzheimer's disease (see McGeer and McGeer (2001) Arch Neurol 58: 1790-2; and De Luigi et al. (2001) Mech Aging Dev 122: 1985-95).

### 4.2. Definitions

For convenience, the meaning of certain terms and phrases employed in the specification, examples, and appended claims is provided below.

The term “allele” refers to the different sequence variants found at different polymorphic regions. For example, IL-1RN (VNTR) has at least five different alleles. The sequence variants may be single or multiple base changes, including without limitation insertions, deletions, or substitutions, or may be a variable number of sequence repeats.

The term “allelic pattern” refers to the identity of an allele or alleles at one or more polymorphic regions. For example, an allelic pattern may consist of a single allele at a polymorphic site, as for IL-1RN (VNTR) allele 1, which is an allelic pattern having at least one copy of IL-1RN allele 1 at the VNTR of the IL-1RN gene loci. Alternatively, an allelic pattern may consist of either a homozygous or heterozygous state at a single polymorphic site. For example, IL1-RN (VNTR) allele 2,2 is an allelic pattern in which there are two copies of the second allele at the VNTR marker of IL-1RN that corresponds to the homozygous IL-RN (VNTR) allele 2 state. Alternatively, an allelic pattern may consist of the identity of alleles at more than one polymorphic site.

The term “antibody ” as used herein is intended to refer to a binding agent including a whole antibody or a binding fragment thereof which is specifically reactive with an IL-1 polypeptide. Antibodies can be fragmented using conventional techniques and the fragments screened for utility in the same manner as described above for whole antibodies. For example, F(ab)2 fragments can be generated by treating an antibody with pepsin. The resulting F(ab)2 fragment can be treated to reduce disulfide bridges to produce Fab fragments. The antibody of the present invention is further intended to include bispecific, single-chain, and chimeric and humanized molecules having affinity for an IL-1B polypeptide conferred by at least one CDR region of the antibody.

“Biological activity” or “bioactivity” or “activity” or “biological function”, which are used interchangeably, for the purposes herein means an effector or antigenic function that is directly or indirectly performed by an IL-1 polypeptide (whether in its native or denatured conformation), or by any subsequence thereof. Biological activities include binding to a target peptide, e.g., an IL-1 receptor. An IL-1 bioactivity can be modulated by directly affecting an IL-1 polypeptide. Alternatively, an IL-1 bioactivity can be modulated by modulating the level of an IL-1 polypeptide, such as by modulating expression of an IL-1 gene.

As used herein the term “bioactive fragment of an IL-1 polypeptide” refers to a fragment of a full-length IL-1 polypeptide, wherein the fragment specifically mimics or antagonizes the activity of a wild-type IL-1 polypeptide. The bioactive fragment preferably is a fragment capable of interacting with an interleukin receptor.

The term “an aberrant activity”, as applied to an activity of a polypeptide such as IL-1, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example an aberrant polypeptide can interact with a different target peptide. A cell can have an aberrant IL-1 activity due to overexpression or underexpression of an IL-1 locus gene encoding an IL-1 locus polypeptide.

“Cells”, “host cells” or “recombinant host cells” are terms used interchangeably herein to refer not only to the particular subject cell, but to the progeny or potential progeny of such a cell. Because certain modifications may occur in succeeding generations due to either mutation or environmental influences, such progeny may not, in fact be identical to the parent cell, but are still included within the scope of the term as used herein.

A “chimera,” “mosaic,” “chimeric mammal” and the like, refers to a transgenic mammal with a knock-out or knock-in construct in at least some of its genome-containing cells.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion of the IL-1 gene cluster. However, where the sample to be tested is genomic DNA, the control sample is preferably a highly purified sample of genomic DNA.

The phrase “diseases and conditions associated with IL-1 polymorphisms” refers to a variety of diseases or conditions, the susceptibility to which can be indicated in a subject based on the identification of one or more alleles within the IL-1 complex. Examples include: inflammatory or degenerative disease, including: Systemic Inflammatory Response (SIRS); Alzheimer's Disease (and associated conditions and symptoms including: chronic neuroinflammation, glial activation; increased microglia; neuritic plaque formation; and response to therapy); Amylotropic Lateral Sclerosis (ALS), arthritis (and associated conditions and symptoms including: acute joint inflammation, antigen-induced arthritis, arthritis associated with chronic lymphocytic thyroiditis, collagen-induced arthritis, juvenile chronic arthritis; juvenile rheumatoid arthritis, osteoarthritis, prognosis and streptococcus-induced arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, glomerulonephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyperresponsiveness), Ophthalmopathies (and associated diseases and conditions, including diabetic retinopathy, Graves' Ophthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis; rheumatism including rheumatoid arthritis, rheumatic aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Immunological disorders, including autoimmune diseases, such as alopecia aerata, autoimmune myocarditis, Graves' disease, Graves ophthalmopathy, lichen sclerosis, multiple sclerosis, psoriasis, systemic lupus erythematosus, systemic sclerosis, thyroid diseases (e.g. goiter and struma lymphomatosa (Hashimoto's thyroiditis, lymphadenoid goiter), sleep disorders and chronic fatigue syndrome and obesity (non-diabetic or associated with diabetes). Resistance to infectious diseases, such as Leishmaniasis, Leprosy, Lyme Disease, Lyme Carditis, malaria, cerebral malaria, meningititis, tubulointestitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosomes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries). Susceptibility to neoplasias, including breast cancer associated osteolytic metastasis, cachexia, colorectal cancer, hyperproliferative diseases, Hodgkin's disease, leukemias, lymphomas, metabolic diseases and tumors, metastases, myeolomas, and various cancers (including breast prostate ovarian, colon, lung, etc), anorexia and cachexia. Hormonal regulation including fertility/fecundity, likelihood of a pregnancy, incidence of preterm labor, prenatal and neonatal complications including preterm low birth weight, cerebral palsy, septicemia, hypothyroxinernia, oxygen dependence, cranial abnormality, early onset menopause. A subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

The term “haplotype” as used herein is intended to refer to a set of alleles that are inherited together as a group (are in linkage disequilibrium) at statistically significant levels (p_{corr}<0.05). As used herein, the phrase “an IL-1 haplotype” refers to a haplotype in the IL-1 loci. An IL-1 inflammatory or proinflammatory haplotype refers to a haplotype that is indicative of increased agonist and/or decreased antagonist activities.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

The terms “IL-1 gene cluster” and “IL-1 loci” as used herein include all the nucleic acid at or near the 2q13 region of chromosome 2, including at least the IL-1A, IL-1B and IL-1RN genes and any other linked sequences. (Nicklin et al., Genomics 19: 382-84, 1994). The terms “IL-1A”, “IL-1B”, and “IL-1RN” as used herein refer to the genes coding for IL-1, IL-1, and IL-1 receptor antagonist, respectively. The gene accession number for IL-1A, IL-1B, and IL-1RN are X03833, X04500, and X64532, respectively.

“IL-1 functional mutation” or “causative mutation” refers to a mutation within the IL-1 gene cluster that results in an altered phenotype (i.e. effects the function of an IL-1 gene or protein). Examples include: IL-1A (+4845) allele 2, IL-1B (+3954) allele 2, IL-1B (+6912) allele 2 and IL-1RN (+2018) allele 2.

“IL-1X (Z) allele Y” refers to a particular allelic form, designated Y, occurring at an IL-1 locus polymorphic site in gene X, wherein X is IL-1A, B, or RN and positioned at or near nucleotide Z, wherein nucleotide Z is numbered relative to the major transcriptional start site, which is nucleotide +1, of the particular IL-1 gene X. As further used herein, the term “IL-1X allele (Z)” refers to all alleles of an IL-1 polymorphic site in gene X positioned at or near nucleotide Z. For example, the term “IL-1RN (+2018) allele” refers to alternative forms of the IL-1RN gene at marker +2018. “IL-1RN (+2018) allele 1” refers to a form of the IL-1RN gene which contains a cytosine (C) at position +2018 of the sense strand. Clay et al., Hum. Genet. 97:723-26, 1996. “IL-1RN (+2018) allele 2” refers to a form of the IL-1RN gene which contains a thymine (T) at position +2018 of the plus strand. When a subject has two identical IL-1RN alleles, the subject is said to be homozygous, or to have the homozygous state. When a subject has two different IL-1RN alleles, the subject is said to be heterozygous, or to have the heterozygous state. The term “IL-1RN (+2018) allele 2,2” refers to the homozygous IL-1RN (+2018) allele 2 state. Conversely, the term “IL-1RN (+2018) allele 1,1” refers to the homozygous IL-1RN (+2018) allele 1 state. The term “IL-1RN (+2018) allele 1,2” refers to the heterozygous allele 1 and 2 state.

“IL-1 related” as used herein is meant to include all genes related to the human IL-1 locus genes on human chromosome 2 (2q 12-14). These include IL-1 genes of the human IL-1 gene cluster located at chromosome 2 (2q 13-14) which include: the IL-1A gene which encodes interleukin-1α, the IL-1B gene which encodes interleukin-1β, and the IL-1RN (or IL-1ra) gene which encodes the interleukin-1 receptor antagonist. Furthermore these IL-1 related genes include the type I and type II human IL-1 receptor genes located on human chromosome 2 (2q12) and their mouse homologs located on mouse chromosome 1 at position 19.5 cM. Interleukin-1α, interleukin-1β, and interleukin-1RN arc related in so much as they all bind to IL-1 type I receptors, however only interleukin-1α and interleukin-1β are agonist ligands which activate IL-1 type I receptors, while interleukin-1RN is a naturally occurring antagonist ligand. Where the term “IL-1” is used in reference to a gene product or polypeptide, it is meant to refer to all gene products encoded by the interleukin-1 locus on human chromosome 2 (2q 12-14) and their corresponding homologs from other species or functional variants thereof. The term IL-1 thus includes secreted polypeptides which promote an inflammatory response, such as IL-1α and IL-1β, as well as a secreted polypeptide which antagonize inflammatory responses, such as IL-1 receptor antagonist and the IL-1 type II (decoy) receptor.

An “IL-1 receptor” or “IL-1R” refers to various cell membrane bound protein receptors capable of binding to and/or transducing a signal from an IL-1 locus-encoded ligand. The term applies to any of the proteins which are capable of binding interleukin-1 (IL-1) molecules and, in their native configuration as mammalian plasma membrane proteins, presumably play a role in transducing the signal provided by IL-1 to a cell. As used herein, the term includes analogs of native proteins with IL-1-binding or signal transducing activity. Examples include the human and murine IL-1 receptors described in U.S. Pat. No. 4,968,607. The term “IL-1 nucleic acid” refers to a nucleic acid encoding an IL-1 protein.

An “IL-1 polypeptide” and “IL-1 protein” are intended to encompass polypeptides comprising the amino acid sequence encoded by the IL-1 genomic DNA sequences shown in Figures contained herein, or fragments thereof, and homologs thereof and include agonist and antagonist polypeptides.

“Increased risk” refers to a statistically higher frequency of occurrence of the disease or condition in an individual carrying a particular polymorphic allele in comparison to the frequency of occurrence of the disease or condition in a member of a population that does not carry the particular polymorphic allele.

The term “interact” as used herein is meant to include detectable relationships or associations (e.g. biochemical interactions) between molecules, such as interactions between protein-protein, protein-nucleic acid, nucleic acid-nucleic acid and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject IL-1 polypeptides preferably includes no more than 10 kilobases (kb) of nucleic acid sequence which naturally immediately flanks the IL-1 gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.).

A “knock-out construct” refers to a nucleic acid sequence that can be used to decrease or suppress expression of a protein encoded by endogenous DNA sequences in a cell. In a simple example, the knock-out construct is comprised of a gene, such as the IL-1RN gene, with a deletion in a critical portion of the gene, so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: IL-1RN 5′/neo/IL-1RN 3′, where IL-1RN5′ and IL-1RN 3′, refer to genomic or cDNA sequences which are, respectively, upstream and downstream relative to a portion of the IL-1RN gene and where neo refers to a neomycin resistance gene. In another knock-out construct, a second selectable marker is added in a flanking position so that the gene can be represented as: IL-1RN/neo/IL-1RN/TK, where TK is a thymidine kinase gene which can be added to either the IL-1RN5′ or the IL-1RN3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern. An example of linkage disequilibrium is that which occurs between the alleles at the IL-1RN (+2018) and IL-1RN (VNTR) polymorphic sites. The two alleles at IL-1RN (+2018) are 100% in linkage disequilibrium with the two most frequent alleles of IL-1RN (VNTR), which are allele 1 and allele 2.

The term “marker” refers to a sequence in the genome that is known to vary among individuals. For example, the IL-1RN gene has a marker that consists of a variable number of tandem repeats (VNTR).

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

A “non-human animal” of the invention includes mammals such as rodents, non-human primates, sheep, dogs, cows, goats, etc. amphibians, such a s members of the Xenopus genus, and transgenic avians (e.g. chickens, birds, etc.). The term “chimeric animal” is used herein to refer to animals in which the recombinant gene is found, or in which the recombinant gene is expressed in some but not all cells of the animal. The term “tissue-specific chimeric animal” indicates that one of the recombinant IL-1 genes is present and/or expressed or disrupted in some tissues but not others. The term “n n-human mammal” refers to any member of the class Mammalia, except for humans.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long.

The term “propensity to disease,” also “predisposition” or “susceptibility” to disease or any similar phrase, means that certain alleles are hereby discovered to be associated with or predictive of a subject's incidence of developing a particular disease (e.g. a vascular disease). The alleles are thus over-represented in frequency in individuals with disease as compared to healthy individuals. Thus, these alleles can be used to predict disease even in pre-symptomatic or pre-diseased individuals.

“Small molecule” as used herein, is meant to refer to a composition, which has a molecular weight of less than about 5 kD and most preferably less than about 4 kD. Small molecules can be nucleic acids, peptides, peptidomimetics, carbohydrates, lipids or other organic or inorganic molecules.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used throughout the specification to refer to DNA sequences, such as initiation signals, enhancers, and promoters, which induce or control transcription of protein coding sequences with which they are operably linked.

As used herein, the term “transgene” means a nucleic acid sequence (encoding, e.g., one of the IL-1 polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of an IL-1 polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques. The term is intended to include all progeny generations. Thus, the founder animal and all F1, F2, F3, and so on, progeny thereof are included.

The term “treating” as used herein is intended to encompass curing as well as ameliorating at least one symptom of a condition or disease.

The term “vector” refers to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

### 4.3. Predictive Medicine

### 4.3.1. IL-1 Inflammatory Haplotypes and Associated Diseases and Conditions.

The present invention is based at least in part, on the identification of certain inflammatory haplotype patterns, particularly those including an IL-1B (−3737) polymorphic allele, and the association (to a statistically significant extent) of these patterns with the development of certain diseases or conditions. Therefore, detection of the alleles comprising a haplotype, alone or in conjunction with another means in a subject can indicate that the subject has or is predisposed to the development of a particular disease or condition. However, because these alleles are in linkage disequilibrium with other alleles, the detection of such other linked alleles can also indicate that the subject has or is predisposed to the development of a particular disease or condition. For example, the 44112332 haplotype comprises the following genotype:

allele 4 of the 222/223 marker of IL-1A
allele 4 of the gz5/gz6 marker of IL-1A
allele 1 of the −889 marker of IL-1A
allele 1 of the +3954 marker of IL-1B
allele 2 of the −511 marker of IL-1B
allele 3 of the gaat.p33330 marker
allele 3 of the Y31 marker
allele 2 of +2018 of IL-1RN
allele 1 of +4845 of IL-1A
allele 2 of the VNTR marker of IL-1RN

Three other polymorphisms in an IL-1RN alternative exon (Exon 1ic, which produces an intracellular form of the gene product) are also in linkage disequilibrium with allele 2 of IL-1RN (VNTR) (Clay et al., (1996) Hum Genet 97:723-26). These include: IL-1RN exon 1ic (1812) (GenBank:X77090 at 1812); the IL-1RN exon 1ic (1868) polymorphism (GenBank:X77090 at 1868); and the IL-1RN exon 1ic (1887) polymorphism (GenBank:X77090 at 1887). Furthermore yet another polymorphism in the promoter for the alternatively spliced intracellular form of the gene, the Pic (1731) polymorphism (GenBank:X77090 at 1731), is also in linkage disequilibrium with allele 2 of the IL-1RN (VNTR) polymorphic locus. For each of these polymorphic loci, the allele 2 sequence variant has been determined to be in linkage disequilibrium with allele 2 of the IL-1RN (VNTR) locus (Clay et al., (1996) Hum Genet 97:723-26).

The 33221461 haplotype comprises the following genotype:

allele 3 of the 222/223 marker of IL-1A
allele 3 of the gz5/gz6 marker of IL-1A
allele 2 of the −889 marker of IL-1A
allele 2 of the +3954 marker of IL-1B
allele 1 of the −511 marker of IL-1B
allele 4 of the gaat.p33330 marker
allele 6 of the Y31 marker
allele 1 of +2018 of IL-1RN
allele 2 of +4845 of IL-1A
allele 1 of the VNTR marker of IL-1RN

Individuals with the 44112332 haplotype are typically overproducers of both IL-1α and IL-1β proteins, upon stimulation. In contrast, individuals with the 33221461 haplotype are typically underproducers of IL-1ra. Each haplotype results in a net proinflammatory response. Each allele within a haplotype may have an effect, as well as a composite genotype effect. In addition, particular diseases may be associated with both haplotype patterns.

The following Table 1 sets forth a number of genotype markers and various diseases and conditions to which these markers have been found to be associated to a statistically significant extent.

TABLE 1
Association Of IL-1 Haplotype Gene
Markers With Certain Diseases

| | GENOTYPE | | | | |
|---|---|---|---|---|---|
| | IL-1A | IL-1A | IL-1B | IL-1B | IL-1RN |
| DISEASE | (−889) | (+4845) | (−511) | (+3954) | (+2018) |
| Periodontal Disease | (*2) | *2 | | *2 | |
| Coronary Artery Disease | | | *2 | | *2 |
| Atherosclerosis | | | | | |
| Osteoporosis | | | | | *2 |
| Insulin dependent | | | | *2 | |
| diabetes | | | | | |
| Diabetic retinopathy | | | | | *1 |
| Endstage renal diseases | | | | | (+) |
| Diabetic nephropathy | | | | | *2 |
| Hepatic fibrosis | | | | | (+) |
| (Japanese alcoholics) | | | | | |
| Alopecia areata | | | | | *2 |
| Graves' disease | | | | | *2 |
| Graves' ophthalmopathy | | | | | (−) |
| Extrathyroid disease | | | | | (+) |
| Systemic Lupus | | | | | *2 |
| Erythematosus | | | | | |
| Lichen Sclerosis | | | | | *2 |
| Arthritis | | | | | (+) |
| Juvenile chronic arthritis | *2 | | | | |
| Rheumatoid arthritis | | | | | (+) |
| Insulin dependent | | | | *2 | *2 |
| diabetes | | | | | VNTR |
| Ulcerative colitis | | | | | *2 |
| Asthma | | | *2 | *2 | |
| Multiple sclerosis | | | | (*2) | *2 |
| | | | | | VNTR |
| Menopause, early onset | | | | | *2 |

In addition to the allelic patterns described above, as described herein, one of skill in the art can readily identify other alleles (including polymorphisms and mutations) that are in linkage disequilibrium with an allele associated with a disease or disorder. For example, a nucleic acid sample from a first group of subjects without a particular disorder can be collected, as well as DNA from a second group of subjects with the disorder. The nucleic acid sample can then be compared to identify those alleles that are over-represented in the second group as compared with the first group, wherein such alleles are presumably associated with a disorder, which is caused or contributed to by inappropriate interleukin 1 regulation. Alternatively, alleles that are in linkage disequilibrium with an allele that is associated with the disorder can be identified, for example, by genotyping a large population and performing statistical analysis to determine which alleles appear more commonly together than expected. Preferably the group is chosen to be comprised of genetically related individuals. Genetically related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms which are linked along a chromosome in a founder population to redistribute through genetic cross-over events. Thus race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-causing mutation is a meta-stable state. Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

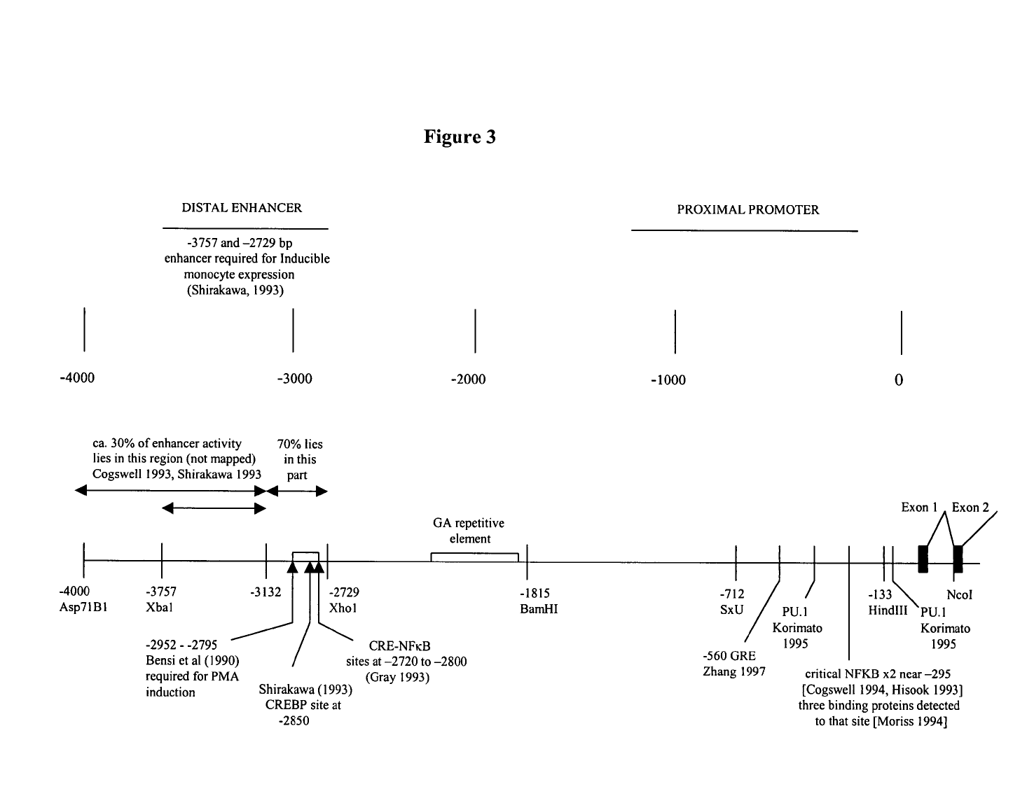

Appropriate probes may be designed to hybridize to a specific gene of the IL-1 locus, such as IL-1A, IL-1B or IL-1RN or a related gene. These genomic DNA sequences are known in the art and available at www.ncbi.nlm.nih gov. shown in FIGS. 3, 4 and 5, respectively, and further correspond to SEQ ID Nos. 1, 2 and 3, respectively. Indeed the IL-1 region of human chromosome 2 spans some 400,000 base pairs and, assuming an average of one single nucleotide polymorphism every 1,000 base pairs, includes some 400 SNPs loci alone. Yet other polymorphisms available for use with the immediate invention are obtainable from various public sources. For example, the human genome database collects intragenic SNPs, is searchable by sequence and currently contains approximately 2,700 entries (http://hgbase.interactiva.de). Also available is a human polymorphism database maintained by the Massachusetts Institute of Technology (MIT SNP database (http://www.genome.wi.mit.edu/SNP/human/index.html)). From such sources SNPs as well as other human polymorphisms may be found.

For example, examination of the IL-1 region of the human genome in any one of these databases reveals that the IL-1 locus genes are flanked by a centromere proximal polymorphic marker designated microsatellite marker AFM220ze3 at 127.4 cM (centiMorgans) (see GenBank Acc. No. Z17008) and a distal polymorphic marker designated microsatellite anchor marker AFM087xa1 at 127.9 cM (see GenBank Acc. No. Z16545). These human polymorphic loci are both CA dinucleotide repeat microsatellite polymorphisms, and, as such, show a high degree of heterozygosity in human populations. For example, one allele of AFM220ze3 generates a 211 bp PCR amplification product with a 5′ primer of the sequence TGTACCTAAGCCCACCCTTTAGAGC and a 3′ primer of the sequence TGGCCTCCAGAAACCTCCAA. Furthermore, one allele of AFM087xa1 generates a 177 bp PCR amplification product with a 5′ primer of the sequence GCTGATATTCTGGTGGGAAA and a 3′ primer of the sequence GGCAAGAGCAAAACTCTGTC. Equivalent primers corresponding to unique sequences occurring 5′ and 3′ to these human chromosome 2 CA dinucleotide repeat polymorphisms will be apparent to one of skill in the art. Reasonable equivalent primers include those which hybridize within about 1 kb of the designated primer, and which further are anywhere from about 17 bp to about 27 bp in length. A general guideline for designing primers for amplification of unique human chromosomal genomic sequences is that they possess a melting temperature of at least about 50 C, wherein an approximate melting temperature can be estimated using the formula Tₘₑₗₜ=[2×(# of A or T)+4×(# of G or C)].

A number of other human polymorphic loci occur between these two CA dinucleotide repeat polymorphisms and provide additional targets for determination of a prognostic allele in a family or other group of genetically related individuals. For example, the National Center for Biotechnology Information web site (www.ncbi.nlm.nih.gov/genemap/) lists a number of polymorphism markers in the region of the IL-1 locus and provides guidance in designing appropriate primers for amplification and analysis of these markers.

Accordingly, the nucleotide segments of the invention may be used for their ability to selectively form duplex molecules with complementary stretches of human chromosome 2 q 12-13 or cDNAs from that region or to provide primers for amplification of DNA or cDNA from this region. The design of appropriate probes for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as a molecular probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15M NaCl at temperatures of about 50 [symbol] C to about 70 [symbol] C. Such selective conditions may tolerate little, if any, mismatch between the probe and the template or target strand.

Other alleles or other indicia of a disorder can be detected or monitored in a subject in conjunction with detection of the alleles described above, for example, identifying vessel wall thickness (e.g. as measured by ultrasound), or whether the subject smokes, drinks is overweight, is under stress or exercises.

### 4.3.2. Detection of Alleles

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express an IL-1 gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of an IL-1 proinflammatory haplotype and having about 5, 10, 20, 25, or 30 nucleotides around the mutation or polymorphic region. In a preferred embodiment of the invention, several probes capable of hybridizing specifically to other allelic variants involved in a restenosis are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of an IL-1 proinflammatory haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of an IL-1 proinflammatory haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed by Maxim and Gilbert ((1977) Proc. Natl Acad Sci USA 74:560) or Sanger (Sanger et at (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of an IL-1 locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify an IL-1 locus allele. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control IL-1 locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of an IL-1 locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition for developing a restenosis. This kit may contain one or more oligonucleotides, including 5′ and 3′ oligonucleotides that hybridize 5′ and 3′ to at least one allele of an IL-1 locus haplotype. PCR amplification oligonucleotides should hybridize between 25 and 2500 base pairs apart, preferably between about 100 and about 500 bases apart, in order to produce a PCR product of convenient size for subsequent analysis.

Particularly preferred primers for use in the diagnostic method of the invention include: TCTAGACCAGGGAGGAGAATGGAATGT [symbol] CCTTGGACTCTGCATGT, and TCTAGACCAGGGAGGAGAATGGAATGT [symbol] CCTTGGACTCTGCATGT for the detection of an IL-1B (−3737) polymorphic allele; ACAGAGGCTCACTCCCTTGCATAATGCAGAGCGAGCACGATACCTGG, and ACAGAGGCTCACTCCCTTGTATAATGCAGAGCGAGCACGATACCTGG for the detection of an IL-1B (−1469) polymorphic allele; and GATCGTGCCACTgcACTCCAGCCTGGGCGACAGGGTGAGACTCTGTCTC, and GATCGTGCCACTgcACTCCAGCCTGGGCGACAGCGTGAGACTCTGTCTC for the detection of an IL-1B (−999) polymorphic allele.

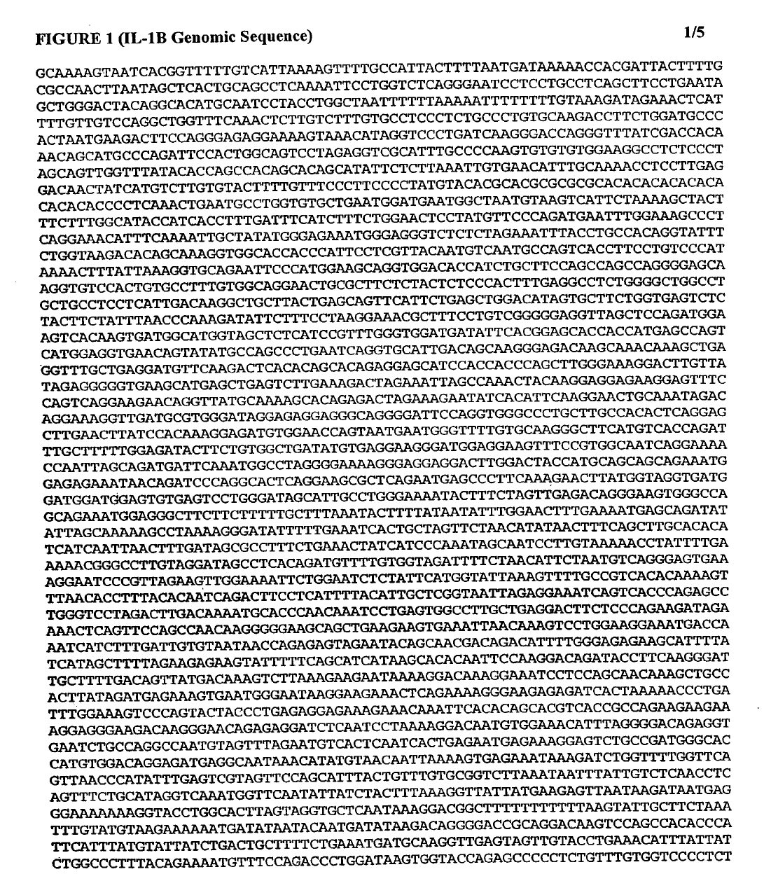

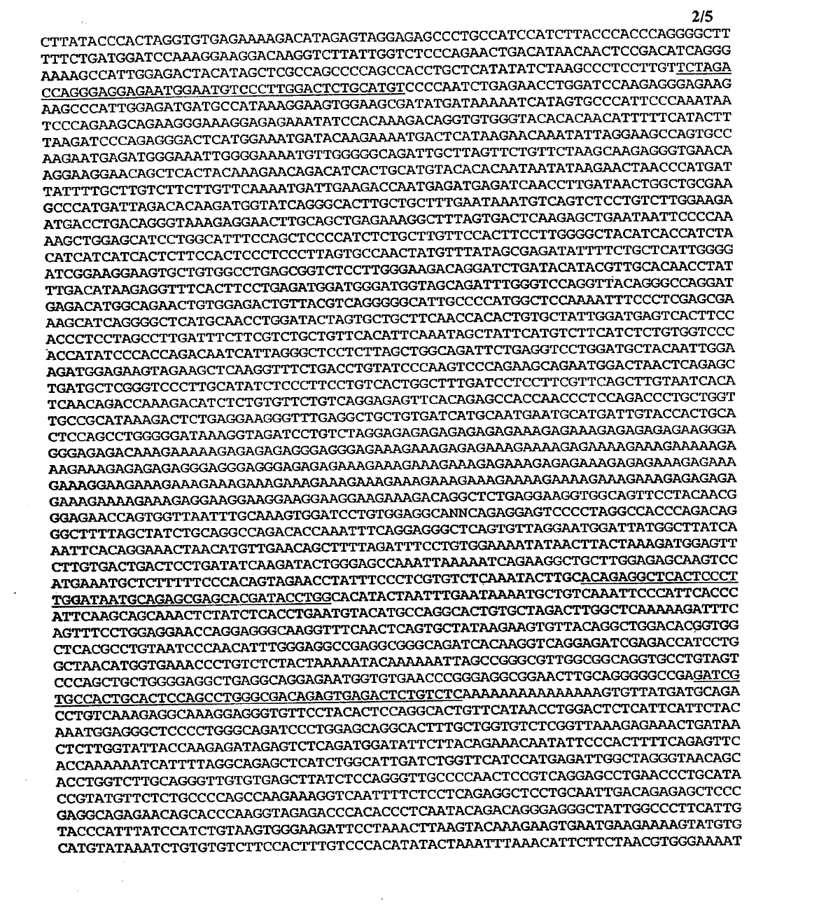

The design of additional oligonucleotides for use in the amplification and detection of IL-1 polymorphic alleles by the method of the invention is facilitated by the availability of both updated sequence information from human chromosome 2q13—which contains the human IL-1 locus, and updated human polymorphism information available for this locus. For example, the DNA sequence for the IL-1A, IL-1B and IL-1RN is shown in FIG. 1 (GenBank Accession No. X03833), 2 (GenBank Accession No. X04500) and 3 (GenBank Accession No. X64532) respectively. Suitable primers for the detection of a human polymorphism in these genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin M. H. J., Weith A. Duff G. W., “A Physical Map of the Region Encompassing the Human Interleukin-1α, interleukin-1β, and Interleukin-1 Receptor Antagonist Genes” Genomics 19: 382 (1995); Nothwang H. G., et al. “Molecular Cloning of the Interleukin-1 gene Cluster: Construction of an Integrated YAC/PAC Contig and a partial transcriptional Map in the Region of Chromosome 2q13” Genomics 41: 370 (1997); Clark, et al. (1986) Nucl. Acids. Res., 14:7897-7914 [published erratum appears in Nucleic Acids Res., 15:868 (1987) and the Genome Database (GDB) project at the URL http://www.gdb.org).

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dermatol. 103:387-389 (1994)) and the like; DNA purification reagents such as Nucleon™ kits, lysis buffers, proteinase solutions and the like; PCR reagents, such as 10× reaction buffers, thermostable polymerase, dNTPs, and the like; and allele detection means such as the HinfI restriction enzyme, allele specific oligonucleotides, degenerate oligonucleotide primers for nested PCR from dried blood.

### 4.3.3. Pharmacogenomics

Knowledge of the particular alleles associated with a susceptibility to developing a particular disease or condition, alone or in conjunction with information on other genetic defects contributing to the particular disease or condition allows a customization of the prevention or treatment in accordance with the individual's genetic profile, the goal of “pharmacogenomics”. Thus, comparison of an individual's IL-1 profile to the population profile for a vascular disorder, permits the selection or design of drugs or other therapeutic regimens that are expected to be safe and efficacious for a particular patient or patient population (i.e., a group of patients having the same genetic alteration).

In addition, the ability to target populations expected to show the highest clinical benefit, based on genetic profile can enable: 1) the repositioning of already marketed drugs; 2) the rescue of drug candidates whose clinical development has been discontinued as a result of safety or efficacy limitations, which are patient subgroup-specific; and 3) an accelerated and less costly development for candidate therapeutics and more optimal drug labeling (e.g. since measuring the effect of various doses of an agent on the causative mutation is useful for optimizing effective dose).

The treatment of an individual with a particular therapeutic can be monitored by determining protein (e.g. IL-1α, IL-1β, or IL-1Ra), mRNA and/or transcriptional level. Depending on the level detected, the therapeutic regimen can then be maintained or adjusted (increased or decreased in dose). In a preferred embodiment, the effectiveness of treating a subject with an agent comprises the steps of: (i) obtaining a preadministration sample from a subject prior to administration of the agent; (ii) detecting the level or amount of a protein, mRNA or genomic DNA in the preadministration sample; (iii) obtaining one or more post-administration samples from the subject; (iv) detecting the level of expression or activity of the protein, mRNA or genomic DNA in the post-administration sample; (v) comparing the level of expression or activity of the protein, mRNA or genomic DNA in the preadministration sample with the corresponding protein, mRNA or genomic DNA in the postadministration sample, respectively; and (vi) altering the administration of the agent to the subject accordingly.

Cells of a subject may also be obtained before and after administration of a therapeutic to detect the level of expression of genes other than an IL-1 gene to verify that the therapeutic does not increase or decrease the expression of genes which could be deleterious. This can be done, e.g., by using the method of transcriptional profiling. Thus, mRNA from cells exposed in vivo to a therapeutic and mRNA from the same type of cells that were not exposed to the therapeutic could be reverse transcribed and hybridized to a chip containing DNA from numerous genes, to thereby compare the expression of genes in cells treated and not treated with the therapeutic.

4.4. Therapeutics for Diseases and Conditions Associated with IL-1 Polymorphisms

Therapeutic for diseases or conditions associated with an IL-1 polymorphism or haplotype refers to any agent or therapeutic regimen (including pharmaceuticals, nutraceuticals and surgical means) that prevents or postpones the development of or alleviates the symptoms of the particular disease or condition in the subject. The therapeutic can be a polypeptide, peptidomimetic, nucleic acid or other inorganic or organic molecule, preferably a “small molecule” including vitamins, minerals and other nutrients. Preferably the therapeutic can modulate at least one activity of an IL-1 polypeptide, e.g., interaction with a receptor, by mimicking or potentiating (agonizing) or inhibiting (antagonizing) the effects of a naturally-occurring polypeptide. An agonist can be a wild-type protein or derivative thereof having at least one bioactivity of the wild-type, e.g., receptor binding activity. An agonist can also be a compound that upregulates expression of a gene or which increases at least one bioactivity of a protein. An agonist can also be a compound which increases the interaction of a polypeptide with another molecule, e.g., a receptor. An antagonist can be a compound which inhibits or decreases the interaction between a protein and another molecule, e.g., a receptor or an agent that blocks signal transduction or post-translation processing (e.g., IL-1 converting enzyme (ICE) inhibitor). Accordingly, a preferred antagonist is a compound which inhibits or decreases binding to a receptor and thereby blocks subsequent activation of the receptor. An antagonist can also be a compound that downregulates expression of a gene or which reduces the amount of a protein present. The antagonist can be a dominant negative form of a polypeptide, e.g., a form of a polypeptide which is capable of interacting with a target peptide, e.g., a receptor, but which does not promote the activation of the receptor. The antagonist can also be a nucleic acid encoding a dominant negative form of a polypeptide, an antisense nucleic acid, or a ribozyme capable of interacting specifically with an RNA. Yet other antagonists are molecules which bind to a polypeptide and inhibit its action. Such molecules include peptides, e.g., forms of target peptides which do not have biological activity, and which inhibit binding to receptors. Thus, such peptides will bind to the active site of a protein and prevent it from interacting with target peptides. Yet other antagonists include antibodies that specifically interact with an epitope of a molecule, such that binding interferes with the biological function of the polypeptide. In yet another preferred embodiment, the antagonist is a small molecule, such as a molecule capable of inhibiting the interaction between a polypeptide and a target receptor. Alternatively, the small molecule can function as an antagonist by interacting with sites other than the receptor binding site.

Modulators of IL-1 (e.g. IL-1α, IL-1β or IL-1 receptor antagonist) or a protein encoded by a gene that is in linkage disequilibrium with an IL-1 gene can comprise any type of compound, including a protein, peptide, peptidomimetic, small molecule, or nucleic acid. Preferred agonists include nucleic acids (e.g. encoding an IL-1 protein or a gene that is up- or down-regulated by an IL-1 protein), proteins (e.g. IL-1 proteins or a protein that is up- or down-regulated thereby) or a small molecule (e.g. that regulates expression or binding of an IL-1 protein). Preferred antagonists, which can be identified, for example, using the assays described herein, include nucleic acids (e.g. single (antisense) or double stranded (triplex) DNA or PNA and ribozymes), protein (e.g. antibodies) and small molecules that act to suppress or inhibit IL-1 transcription and/or protein activity.

### 4.4.1. Affective Dose

Toxicity and therapeutic efficacy of such compounds can be determined by standard pharmaceutical procedures in cell cultures or experimental animals, e.g., for determining The LD50 (the dose lethal to 50% of the population) and the Ed50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissues in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### 4.4.2. Formulation and Use

Compositions for use in accordance with the present invention may be formulated in a conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can he injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### 4.5. Assays to Identify Therapeutics

Based on the identification of mutations that cause or contribute to the development of a disease or disorder that is associated with an IL-1 polymorphism or haplotype, the invention further features cell-based or cell free assays for identifying therapeutics. In one embodiment, a cell expressing an IL-1 receptor, or a receptor for a protein that is encoded by a gene which is in linkage disequilibrium with an IL-1 gene, on the outer surface of its cellular membrane is incubated in the presence of a test compound alone or in the presence of a test compound and another protein and the interaction between the test compound and the receptor or between the protein (preferably a tagged protein) and the receptor is detected, e.g., by using a microphysiometer (McConnell et al. (1992) Science 257:1906). An interaction between the receptor and either the test compound or the protein is detected by the microphysiometer as a change in the acidification of the medium. This assay system thus provides a means of identifying molecular antagonists which, for example, function by interfering with protein-receptor interactions, as well as molecular agonist which, for example, function by activating a receptor.

Cellular or cell-free assays can also be used to identify compounds which modulate expression of an IL-1 gene or a gene in linkage disequilibrium therewith, modulate translation of an mRNA, or which modulate the stability of an mRNA or protein. Accordingly, in one embodiment, a cell which is capable of producing an IL-1, or other protein is incubated with a test compound and the amount of protein produced in the cell medium is measured and compared to that produced from a cell which has not been contacted with the test compound. The specificity of the compound vis a vis the protein can he confirmed by various control analysis, e.g., measuring the expression of one or more control genes. In particular, this assay can be used to determine the efficacy of antisense, ribozyme and triplex compounds.

Cell-free assays can also be used to identify compounds which are capable of interacting with a protein, to thereby modify the activity of the protein. Such a compound can, e.g., modify the structure of a protein thereby effecting its ability to bind to a receptor. In a preferred embodiment, cell-free assays for identifying such compounds consist essentially in a reaction mixture containing a protein and a test compound or a library of test compounds in the presence or absence of a binding partner. A test compound can be, e.g., a derivative of a binding partner, e.g., a biologically inactive target peptide, or a small molecule.

Accordingly, one exemplary screening assay of the present invention includes the steps of contacting a protein or functional fragment thereof with a test compound or library of test compounds and detecting the formation of complexes. For detection purposes, the molecule can be labeled with a specific marker and the test compound or library of test compounds labeled with a different marker. Interaction of a test compound with a protein or fragment thereof can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the protein or functional fragment thereof is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) an IL-1 or other protein, (ii) an appropriate receptor, and (iii) a test compound; and (b) detecting interaction of the protein and receptor. A statistically significant change (potentiation or inhibition) in the interaction of the protein and receptor in the presence of the test compound, relative to the interaction in the absence of the test compound, indicates a potential antagonist (inhibitor). The compounds of this assay can be contacted simultaneously. Alternatively, a protein can first be contacted with a test compound for an appropriate amount of time, following which the receptor is added to the reaction mixture. The efficacy of the compound can be assessed by generating dose response curves from data obtained using various concentrations of the test compound. Moreover, a control assay can also be performed to provide a baseline for comparison. Complex formation between a protein and receptor may be detected by a variety of techniques. Modulation of the formation of complexes can be quantitated using, for example, detectably labeled proteins such as radiolabeled, fluorescently labeled, or enzymatically labeled proteins or receptors, by immunoassay, or by chromatographic detection.

Typically, it will be desirable to immobilize either the protein or the receptor to facilitate separation of complexes from uncomplexed forms of one or both of the proteins, as well as to accommodate automation of the assay. Binding of protein and receptor can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the receptor, e.g. an 35S-labeled receptor, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintillant), or in the supernatant after the complexes are subsequently dissociated. Alternatively, the complexes can be dissociated from the matrix, separated by SDS-PAGE, and the level of protein or receptor found in the bead fraction quantitated from the gel using standard electrophoretic techniques such as described in the appended examples. Other techniques for immobilizing proteins on matrices are also available for use in the subject assay. For instance, either protein or receptor can be immobilized utilizing conjugation of biotin and streptavidin. Transgenic animals can also be made to identify agonists and antagonists or to confirm the safety and efficacy of a candidate therapeutic. Transgenic animals of the invention can include non-human animals containing a restenosis causative mutation under the control of an appropriate endogenous promoter or under the control of a heterologous promoter.

The transgenic animals can also be animals containing a transgene, such as reporter gene, under the control of an appropriate promoter or fragment thereof. These animals are useful, e.g., for identifying drugs that modulate production of an IL-1 protein, such as by modulating gene expression. Methods for obtaining transgenic non-human animals are well known in the art. In preferred embodiments, the expression of the restenosis causative mutation is restricted to specific subsets of cells, tissues or developmental stages utilizing, for example, cis-acting sequences that control expression in the desired pattern. In the present invention, such mosaic expression of a protein can be essential for many forms of lineage analysis and can additionally provide a means to assess the effects of, for example, expression level which might grossly alter development in small patches of tissue within an otherwise normal embryo. Toward this end, tissue-specific regulatory sequences and conditional regulatory sequences can be used to control expression of the mutation in certain spatial patterns. Moreover, temporal patterns of expression can be provided by, for example, conditional recombination systems or prokaryotic transcriptional regulatory sequences. Genetic techniques, which allow for the expression of a mutation can be regulated via site-specific genetic manipulation in vivo, are known to those skilled in the art.

The transgenic animals of the present invention all include within a plurality of their cells a causative mutation transgene of the present invention, which transgene alters the phenotype of the “host cell”. In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats. Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation of expression of the causative mutation transgene can be regulated via control of recombinase expression.

Use of the cre/loxP recombinase system to regulate expression of a causative mutation transgene requires the construction of a transgenic animal containing transgenes encoding both the Cre recombinase and the subject protein. Animals containing both the Cre recombinase and the restenosis causative mutation transgene can be provided through the construction of “double” transgenic animals. A convenient method for providing such animals is to mate two transgenic animals each containing a transgene.

Similar conditional transgenes can be provided using prokaryotic promoter sequences which require prokaryotic proteins to be simultaneous expressed in order to facilitate expression of the transgene. Exemplary promoters and the corresponding trans-activating prokaryotic proteins are given in U.S. Pat. No. 4,833,080. Moreover, expression of the conditional transgenes can be induced by gene therapy-like methods wherein a gene encoding the transactivating protein, e.g. a recombinase or a prokaryotic protein, is delivered to the tissue and caused to be expressed, such as in a cell-type specific manner. By this method, the transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for microinjection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote. Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. Further, in such embodiments the sequence will be attached to a transcriptional control element, e.g., a promoter, which preferably allows the expression of the transgene product in a specific type of cell.

Retroviral infection can also be used to introduce the transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

The present invention is further illustrated by the following examples which should not be construed as limiting in any way. The contents of all cited references (including literature references, issued patents, published patent applications as cited throughout this application) are hereby expressly incorporated by reference. The practice of the present invention will employ, unless otherwise indicated, conventional techniques that are within the skill of the art. Such techniques are explained fully in the literature. See, for example, Molecular Cloning A Laboratory Manual, (2nd ed., Sambrook, Fritsch and Maniatis, eds., Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; U.S. Pat. No. 4,683,202; and Nucleic Acid Hybridization (B. D. Hames & S. J. Higgins eds., 1984).

### 5. EXAMPLES

### 5.1. Molecular Analysis of IL-1B (−3737) Polymorphism

In this example, we cloned, sequenced, and analysed the transcription al effects of alleles of a previously unknown upstream polymorphism of the IL-1B gene. We have previously shown a high degree of linkage disequilibrium between markers across the IL-1 gene cluster and this new polymorphism at −3737 is linked to polymorphisms at −511, −31, and +3954 that have previously been associated with altered IL-1 beta production rate, and with susceptibility to inflammatory and infectious diseases. Ascertainment of genotype at this new, functional polymorphism offers a more direct genetic test of susceptibility to diseases where IL-1 production contributes to pathogenesis.

We investigated the transcriptional activity of different alleles of the interleukin-1B (IL-1B) gene. This is of interest because, in North European populations, IL1B allele status is associated with many chronic inflammatory diseases, including periodontitis (Kornman, K. S. et al. (1999), J. Periodontal Res. 34:353), and gastric cancer (El-Omar, E. et al. (2000), Nature 404:398).

Elucidation of the molecular mechanism underlying these associations is important since it would enable the rational design of interventions to modulate the pathological process, and would improve the performance of prognostic genetic testing. Extensive linkage disequilibrium across the IL1 gene cluster (see Cox, A. et al. (1998), Am. J. Hum. Genet. 62: 1180) makes it possible that currently known ‘marker’ polymorphisms are in linkage with others (‘pathogenic polymorphisms’) that are, themselves, causally related to the disease process. The extent of linkage between ‘marker’ and ‘pathogenic’ polymorphisms, which may vary between races, will be an important determinant of the global performance of a genetic test utilising ‘marker’ polymorphisms. This situation might explain the reduced utility of the commercially available ‘PST test’ outside the North European population, Kornman, K. S. et al. (1999), J. Periodontal Res. 34:353; Armitage, G. C. et al. (2000), J. Periodontol 71:164.

Identification of the functional IL-1 SNPs responsible for increased susceptibility to chronic inflammatory diseases (including cardiovascular disease, periodontitis and gastric cancer) is critical to the rational design of interventions to modulate these pathogenic processes as well as to the refinement of prognostic genetic tests. Our study was designed to investigate the influence of polymorphisms on IL1B transcription. El-Omar and colleagues (see El-Omar, E. et al. (2000), Nature 404: 398) who describe an association with the IL1B -31 (TATA box) polymorphism and gastric cancer, suggested that altered transcription factor binding to the TATA box might be responsible for a transcriptional difference of IL1B gene and be causally related to the disease association they observed (gastric cancer). Transcriptional assays were not, however, presented in their paper. This study investigated the transcriptional activity of currently known SNPs of IL-1B as well as the (−3737) IL-1B polymorphism.

We performed transcription rate (nuclear run on) assays measuring IL1B mRNA extension. These experiments were performed on peripheral blood mononuclear cells (PBMC) ex vivo. The leukocytes were stimulated with LPS 1 ug/ml and nuclear extracts were made 2 hours later. The cells were extracted from a range of individuals selected on the basis of their differing genotypes across the IL1B cluster.

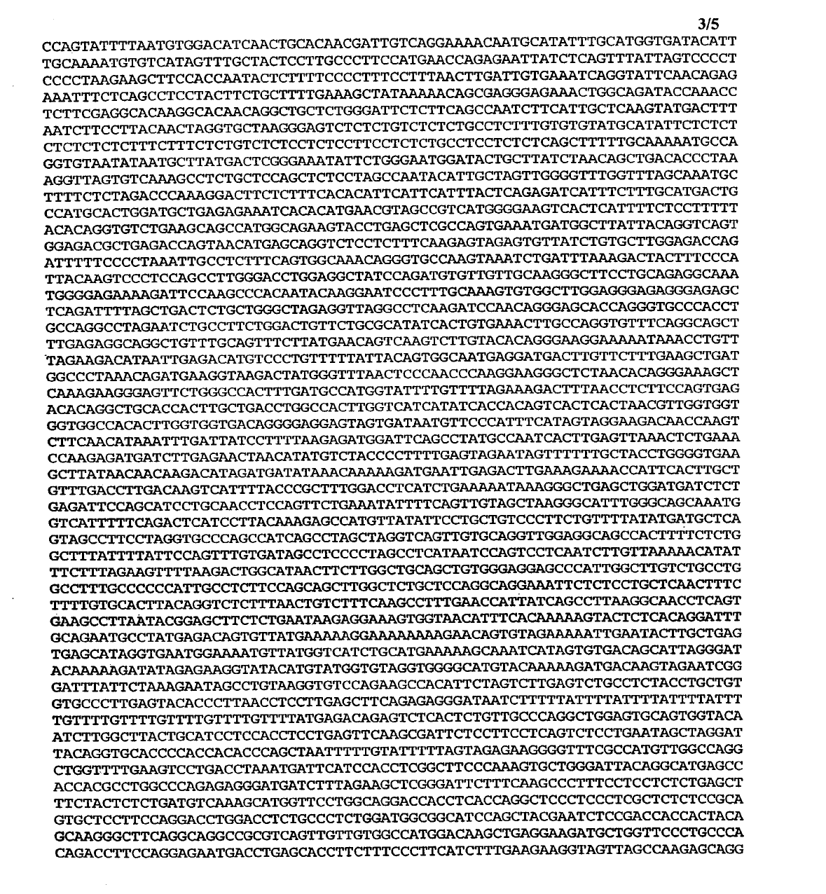

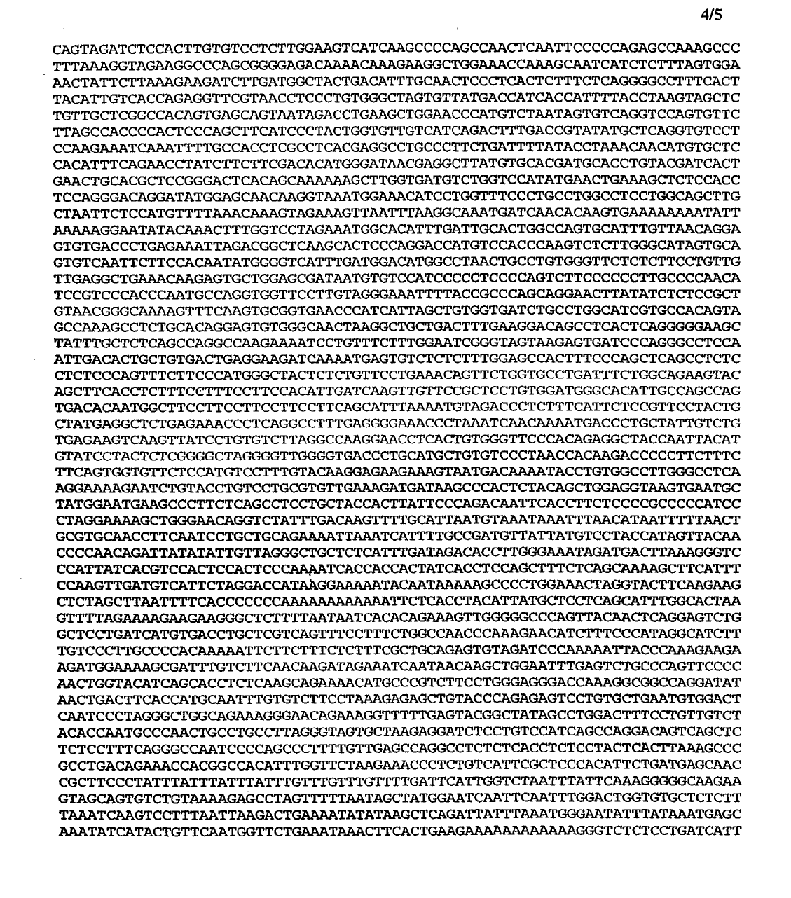

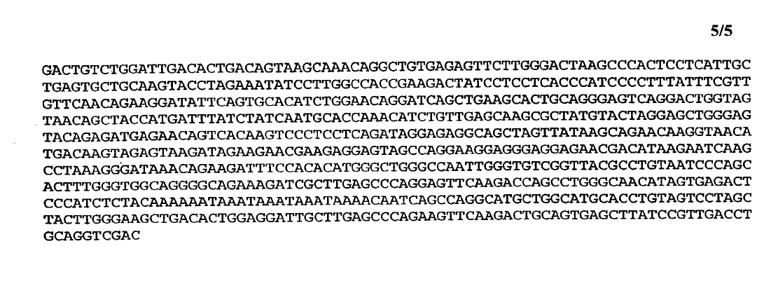

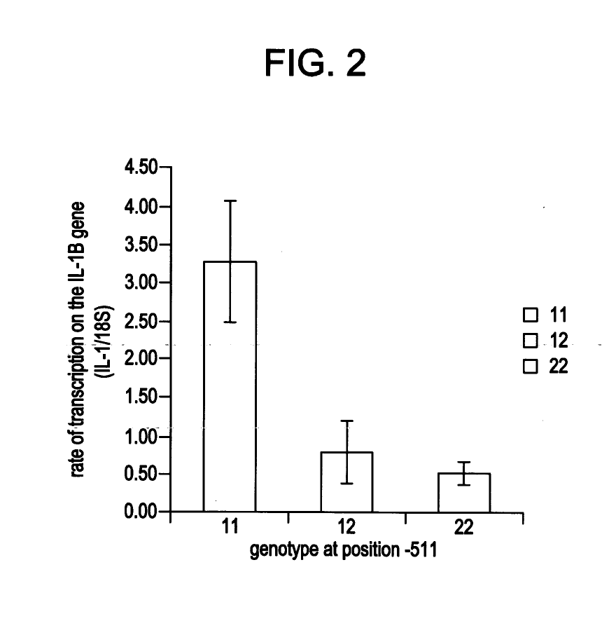

Each individual was studied on three separate occasions and the mean transcriptional activity calculated per individual. This experiment was designed to investigate the effect of the +3953 IL1B polymorphism; no significant differences in IL1B activity were observed associated with this polymorphism. However, when the data was reanalysed to investigate the effect of the −511 polymorphism, allele specific transcriptional differences were evident (see FIG. 2).

The data in FIG. 2 support an association between IL1B transcription and −511 polymorphism status. They do not exclude a contribution of other, linked polymorphisms. The data set may be too small to allow reanalysis by haplotype (see Cox, A. et al. (1998), Am. J. Hum. Genet. 62:1180) although haplotype, rather than individual polymorphisms, have been reported to be associated with several diseases, including rheumatoid arthritis, inflammatory bowel disease severity, and tuberculosis (see e.g. Cox, A. et al. (1998), Am. J. Hum. Genet. 62:1180; Wilkinson, R. J. et al. (1999), J. Exp. Med. 189:1863; Heresbach, D. et al. (1997), Am. J. Gastroenterol. 92:1164; Cox, A. et al. (1999), Hum. Mol. Genet. 8:1707).

### IL1B Promoter Structure

The IL1B promoter is an extensive structure, extending at least 4 kb upstream of the transcription initiator. It is illustrated diagrammatically below (FIG. 3). Several studies in the early 1990s investigated its function by mutagenesis. The strategy was similar in all cases and consisted of ligating promoter fragments to a reporter gene. Exon 1 is non-coding and the ATG lies in exon 2; an NcoI restriction site (CCATGG) encompasses the first codon allowing an easy way to replace the IL1B coding sequence with a reporter gene, retaining exon 1 and the natural splice signals.

These studies demonstrated the presence of two major promoter regions—a proximal one, extending from +547 (the ATG) to ca. −1000 bp, and a distal promoter lying in the region −4000 to −2757 (FIG. 2). This distal promoter is widely referred to in the literature as an ‘enhancer’ (e.g. Bensi, G. et al. (1990), Cell Growth Differ. 1:491; Clark, B. D. et al. (1986) [published erratum appears in Nucleic Acids Res 1987 Jan. 26; 15(2):868], Nucleic Acids Res. 14:7897; Cogswell, J. P. et al. (1994), J. Immunol. 153:712; Shirakawa, F. et al. (1993), Mol. Cell Biol. 13:1332, although orientation independence has not been established experimentally.

Proximal Promoter

The proximal promoter contains multiple potential transcription factor binding sites; NF-kB like elements have been shown experimentally to be important (see Hiscott, J. et al. (1993), Mol. Cell Biol. 13:6231; Monks, B. G. et al. (1994), Mol. Immunol. 31:139; Zhang, Y. and Rom, W. N. (1993), Mol. Cell Biol. 13:3831; Krauer, K. G. et al. (1998), Virology 252:418; Tsukada, J. et al. (1997), Blood 90:3142, NF-IL6 (C/EBP), Shirakawa, F. et al. (1993), Mol. Cell Biol. 13:1332; Zhang, Y. and Rom, W. N. (1993), Mol. Cell Biol. 13:3831; Godambe, S. A. et al. (1994), J. Immunol. 153:143; Godambe, S. A. et al. (1994), DNA Cell Biol. 13:561, and PU-1 like elements, Buras, J. A. et al. (1995), [published erratum appears in Mol Immunol 1995 October; 32(14-15):1175], Mol. Immunol. 32:541; Kominato, Y. et al. (1995), Mol. Cell Biol. 15:59; Lodie, T. A. et al. (1997), J. Immunol. 158:1848; Wara-aswapati, N. et al. (1999), Mol. Cell Biol. 19:6803).

Distal Promoter

The distal promoter consists of a core region (−2982→−2729) (see Bensi, G. et al. (1990), Cell Growth Differ. 1:491) which contains multiple transcription factor binding sites (see Shirakawa, F. et al. (1993), Mol. Cell Biol. 13:1332). This region is required for LPS or PMA induction of IL1B gene in moncytes (Bensi, G. et al. (1990), Cell Growth Differ. 1:491; Shirakawa, F. et al. (1993), Mol. Cell Biol. 13:1332). The C/EBP and NF-kB binding sites in the −2982→−2729 region have been shown experimentally to be functionally important (see Cogswell, J. P. et al. (1994), J. Immunol. 153:712; Shirakawa, F. et al. (1993), Mol. Cell Biol. 13:1332; Gray, J. G. et al. (1993), Mol. Cell Biol. 13:6678). Deletion mutagenesis shows the short −2982→−2729 region of the distal promoter is responsible for ca. 60-70% of the activity of the whole distal promoter region (Cogswell, J. P. et al. (1994), J. Immunol. 153:712; Shirakawa; F. et al. (1993), Mol. Cell Biol. 13:1332) the sequences in the −3753 to −2982 region which are responsible for the remaining ca. 30% have not been defined.

The following experiments address: whether the allele specific transcriptional variation shown above could be demonstrated using reporter constructs; whether the −31 or −511 polymorphisms could be shown to be causally related to transcriptional variation; and whether additional polymorphisms could be discovered which were associated with transcriptional differences. It was accepted that the presence of such regulatory polymorphisms in the region studied would not exclude the presence of other, linked polymorphisms relevant to physiological regulation located outside the studied region.

### Methods

IL1B Containing Cosmid

This cosmid, pCOS-IL1Bus1, was a provided by Dr M. Nicklin in our laboratory. It had been isolated by Dr Nicklin in 1993 from an EMBL genomic DNA library by hybridisation. The ethnic origin of the individual used for the construction of this library is unknown. A restriction map was provided by Dr Nicklin. It was transformed in DII5alpha E. coli and maintained on Kanamycin 50 ug/ml LB agar plates. Amplification was from single colonies at 37 degrees in 20 ml 2× YT medium containing 5 ug/ml Kanamycin.

Reporter Constructs Derived from IL1B Containing Cosmid

A series of these plasmids were constructed. Preliminary experiments showed that the vector pGL3-basic, but not pGL3-enhancer (both from Promega), was suitable for the transfection experiments planned. Initially, the vector pGL3-basic was cut with NcoI and BamHI and the NcoI-BamHI fragment from the cosmid PCOS-IL1BusI containing the proximal IL1B promoter (−1815→+547) ligated in, generating plasmid pILG-A1. Subsequently, a second plasmid was made which included the distal promoter as well. This was constructed by digesting the cosmid pCOS-IL1Bus1 and pILG-A1 with Asp718I and HindIII and ligating the distal promoter −4000 to −1815 into the cut pILG-A1 vector, generating pILG-S1. Digestion of pILG-S1 and pILG-A1 with unique internal restriction sites, followed by filling with Klenow DNA polymerase and intramolecular relegation was used to generate a series of deletion mutants of the IL1B promoter. The plasmids generated thus are shown below in Table 1.

TABLE 1
Plasmids derived from cosmid pCOS-IL1Bus1

| | | Restriction enzyme | |
|---|---|---|---|
| Plasmid | Insert | used | Source plasmid |
| pILG-S1 | −4200 → +547 | Asp718-HindIII, | pCOS-IL1Bus1, |
| | | HindIII-NcoI | pILG-A1 |
| pILG-T1 | −2729 → +547 | Asp718I, XhoI | pILG-S1 |
| pILG-A1 | −1815 → +547 | BamHI-NcoI | pCOS-IL1Bus1, |
| | | | pGL3-basic |
| pILG-E1 | −1604 → +547 | NheI + EcoRV | pILG-A1 |
| pILG-F1 | −1063 → +547 | SmaI | pILG-A1 |
| pILG-G1 | −548 → +547 | BstXI + NheI | pILG-A1 |
| pILG-H1 | −516 → +547 | SacI | pILG-A1 |
| pILG-J1 | −131 → +547 | NheI + HindIII | pILG-A1 |
| pGL3-basic | None | none | Promega |

Mutagenesis of IL1B Promoter

Double stranded automated sequencing was carried out on clone S1. Using the sequence information obtained, oligonucleotides were designed to alter the −511 and −31 residues (see El-Omar, E. et al. (2000), Nature 404:398; and di Giovine, F. S. et al. (1992), Hum. Mol. Genet. 1:450) to the alternative base. These oligonucleotides are designated ‘−31 probe 1’ and ‘−511 probe 1’. The sequences of these oligonucleotides are shown below (and underlined in FIG. 1). They were used to mutagenise the pILG-A1 plasmid using the GeneEditor system (Promega) according to the manufacturer's recommendations. The oligonucleotides were used individually and together in order to produce all possible combinations of −31 and −511 status. Successful mutagenesis, and the absence of secondary mutations, was confirmed by double stranded DNA sequencing.

pILG-A1 derivates contained only the −18154→+547 fragment of the IL1B promoter, so the vectors containing these inserts were digested with Asp718I and XmaI (SmaI) and the pILG-S1 Asp718I→XmaI fragment, which contains a type 2 distal promoter, was ligated onto the mutated proximal promoters. The resulting vectors are shown below in Table 2

TABLE 2
Genotype of mutant type 2 IL1B promoters - mutation
of −31 and −511 sites

| −1815 → +547 | −4000 → +547 | Genotype at −31 | Genotype at −511 |
|---|---|---|---|
| pILG-A1 | pILG-S1 | 2 | 2 |
| pILG-V1 | pILG-AA1 | 1 | 2 |
| pILG-W1 | pILG-AC1 | 2 | 1 |
| pILG-X1 | pILG-AE1 | 1 | 1 |

Extraction of DNA from Human Blood and Cell Lines, and Genotyping

This was performed using a Gentra PureGene blood kit according to the manufacturer's recommendations. The DNA was resuspended in 50 ul of TE buffer and stored at −20. Cells lines were grown as recommended by ATCC, and as follows: HL60, A549 cells, U937, MonoMac6, EHEB-1. All these cell lines are of caucasian origin. 1×10⁷ cells were extracted. DNA was extracted from one human volunteer's PBMC. The only human volunteer used, Dr. Ken Kornman (R&D Director, Interleukin Genetics, Inc.), gave his informed consent for the experiment The genotypes of the cell lines were determined by TaqMan methodology as previously described. Genotypes obtained are shown in Table 3.

TABLE 3
Genotypes of Cell lines Used

| Cell line | −2018 IL1A | −511 IL1B |
|---|---|---|
| KK PBMC DNA | 1.2 | 2.2 |
| EHEB-1 | 1.2 | 1.1 |
| MonoMac6 | 2.2 | 1.2 |
| U937 | Not determined | 1.1 |
| A549 | 1.1 | 2.2 |
| HL60 | 1.1 | 1.2 |

PCR Cloning of Human IL1B Promoter

Conditions for PCR cloning of the human IL1B promoter were optimised. Proof reading enzymes alone (Pfu and Pfx) were investigated but only with proof reading/Taq combinations was product observed. The conditions used ultimately were Trioblock thermocycler, thin walled tubes, oil, 25 ul reactions, 500 pg template, 200 nM dNTPs, 1 mM primers ILG-9 and ILG-18, 1× Herculase polymerase buffer as supplied by the manufacturer (Stratagene). Herculase is a mixture of Pfu-turbo and Taq DNA polymerases. Cycling was as follows: 94 degrees 2 mins, then hot start with 0.5 ul Herculase polymerase, then 30 cycles (94 degrees 30 seconds, 66 degrees 30 seconds, 72 degrees 6 mins). Product was diluted to 50 ul and polymerase and buffer removed using a Chromospin 200 gel filtration column as per the manufacturer's protocol (Clontech). The eluted product was digested with the following enzymes: 10 U Asp718I, 0.02 U NcoI. This achieved partial digestion of the internal and 3′ NcoI sites. The mixture was heat inactivated and ligated into an Asp718I-NcoI digested pGL3-basic vector at appropriate ratios, and transformed into Library efficiency DH5alpha cells (Life Technologies). Positive colonies were identified by PCR screening against the distal enhancer and/or by restriction analysis.

At least two clones of each genotype were obtained from each template. These clones were derived from completely independent PCR reactions, so that PCR mutations, even if occurring early in the PCR cycle could be differentiated from polymorphisms on the basis of their occurrence in multiple isolates.

Plasmids were grown in LB medium. For maxipreparation, 150 ml cultures were used. n and storage was in endotoxin free TE buffer (Qiagen) and tubes (Cryovials, ElutioPlasmid maxipreparation was performed on all plasmids used for transcriptional assays, and used the Qiagen Endofree maxipreparation system, as recommended by the manufacturer, except that the final isopropanol precipitation step was performed in 50 ml endotoxin free disposable centrifuge tubes at 3,500 rpm in a Sanyo swing-out tissue culture centrifuge, a procedure which produced excellent precipitation. Nalgene). Concentrations were determined by UV spectrophotometry on at least two occasions and confirmed by restriction analysis and gel quantification.

Identification of Polymorphisms

Clones were isolated and sequenced by automated sequencing using a set of internal primers designed for the purpose. Sequences were not accepted if >2% ambiguity was present as assessed with the Factura base calling algorithm (ABI). Following ambiguity marking with Factura 1.1, the sequence traces assembled into a single contig with one pass of the AutoAssembler 2.1 (ABI). Manual editing of regions of poor assembly and base calling was performed. The contigs obtained, and annotated chromatograms, are attached on a CD. Consensus was calculated by AutoAssember using default parameters and the sequences obtained aligned and inspected using Genetyx-Mac 7.3 (Software Development Corp.) and/or ClustalX, obtained as freestanding Mac executable from http://www.ncbi.nlm.nih.gov. Polymorphisms were searched for in the aligned sequences by visual inspection, and were considered to be differences between sequences occurring in more than one sequence at the same position. Single base pair differences found in only one sequence were considered to be probable PCR induced mutations and were marked as such.

### Cell Lines

RAW264.7 cells (ECACC 91062702) were grown in RPMI1640 containing penicillin-streptomycin and 10% heat inactivated fetal calf serum. Low endotoxin (<10 mIU/ml) serum was used (Life Technologies). Cells were split by scraping 1:6 (area:area) every 3-4 days.

### Transfection and Transcriptional Assays

RAW264.7 cells were plated into 96 well plates at a density of 2.5×10⁴ cells/well in 100 ul of compete medium. 24 hours later they were transfected with 400 ng of expression vector, which drove the expression of firefly luciferase, and 100 ng of pTK-rLuc (Promega), which drives the expression of Renilla luciferase under a constitutive promoter. 2.5 ul of Superfect (Qiagen) was used to perform this, according to the manufacturer's protocol. The medium/DNA/liposome mixture was aspirated at 2.5 hrs post addition and replaced with 150 ul of prewarmed complete medium. 24 hours subsequently, agonists were added and assay of both luciferase activities (Dual-Luciferase, Promega) performed 6 hrs after addition of agonists. Normalised luciferase activity was expressed as firefly/renilla luciferase light production.

### Results & Discussion

RAW Cells—a Suitable Cell Line for IL1B Study

This study used RAW264 cells, a differentiated macrophage-like cell line, which has previously been shown to be a suitable model for the study of the IL1B promoter. Shirakawa, F. et al. (1993), Mol. Cell Biol. 13:1332. The results show that the distal promoter was required for efficient induction of the IL1B promoter introduced on a plasmid (see Figure).

### Effect of Mutation of −31 or −511 Polymorphisms on Activity of Type 2 Promoter

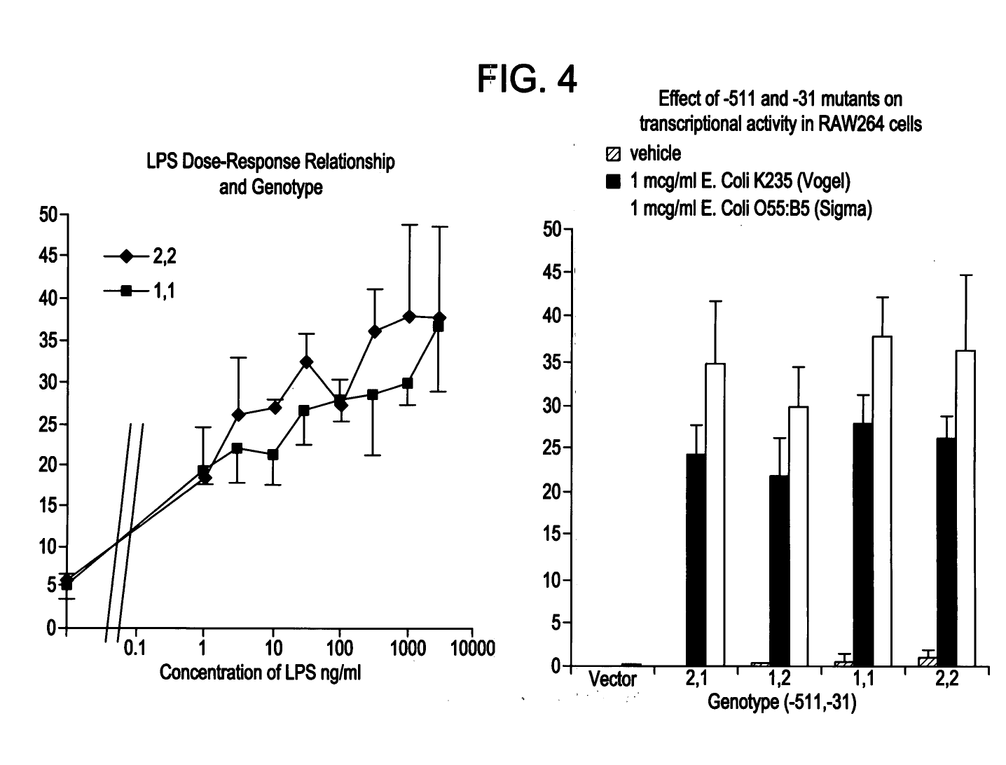

The −31 TATA box polymorphism of the IL promoter has been proposed to be responsible for transcriptional variations between alleles, and consequent pathological effects associated with IL1B phenotype (see El-Omar, E. et al. (2000), Nature 404:398). Such a mechanism has been documented for several other genes (see e.g. Antonarakis, S. E. et al. (1984), Proc. Natl. Acad. Sci. USA 81:1154; Humphries, A. et al. (1999), Blood Cells Mol. Dis. 25:210; Peltoketo, H. et al. (1994), Genomics 23:250; Takihara, Y. et al. (1986), Blood 67:547). The −511 promoter construct obtained from a genomic DNA library, as described in methods, was mutated by site directed mutagenesis to obtain a type 2 construct with all possible combinations of polymorphisms at the −31 and −511 positions. The transcriptional activity attributable to these polymorphisms, individually or in combination, should be discernible by this technique. The converse experiment, in which a type 1 promoter has these sites mutated complements the data with the type 2 promoter shown (see FIG. 4).

FIG. 4 shows a representative experiment of three carried out, in none of which was transcriptional variation associated with −31 or −511 allele status observed. In the left hand panel, the dose-response relationship between concentration of applied LPS and promoter response is shown for mutant (−31=2, −511=2) and wild-type (−31=1, −511=1) promoters. Transcriptional equivalence of the two promoters was evident at all concentrations tested.

Cloning of IL1B Alleles from Different Sources

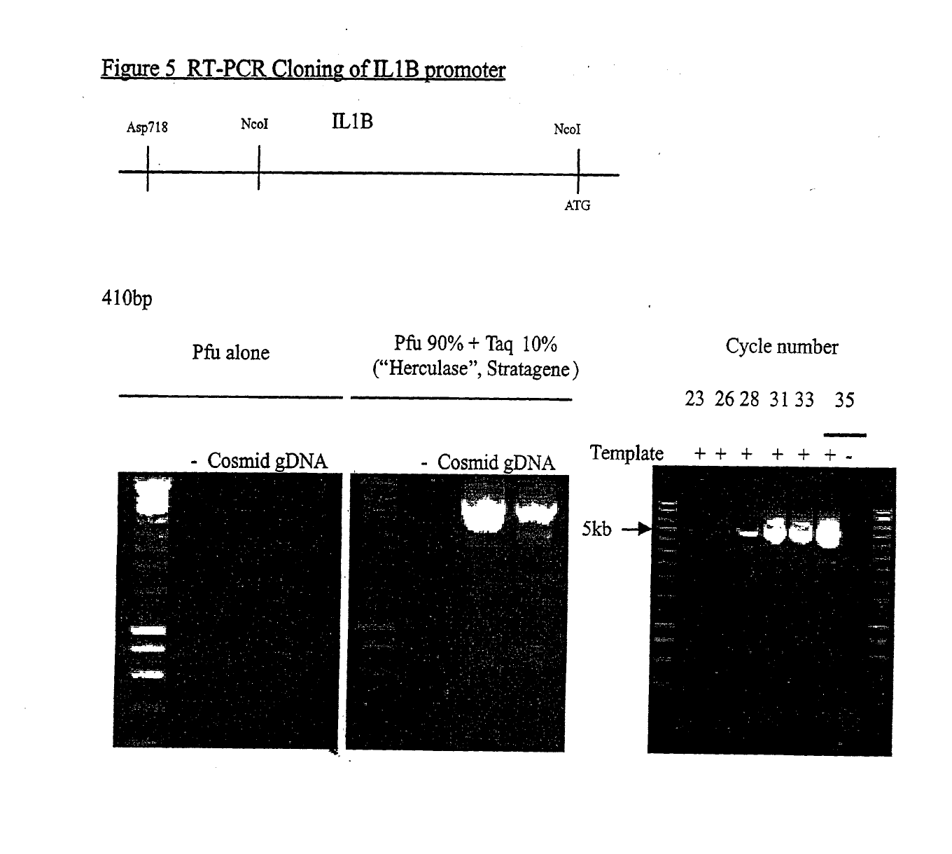

A long distance PCR was used to amplify the IL1B promoter. This required optimisation, but specific amplification was achieved. Initial attempts, which used proofreading polymerases alone, were unsuccessful (sec FIG. 5). To clone the product, the PCR product was digested with Asp718I and NcoI and ligated into the reporter vector pGL3-basic. It was decided not to use a sequence independent cloning method because the yield from these is very low without a selection system to positively select for insert. This can favour odd mutations in unfavorable sequences, and is difficult to control.

### Clones Obtained by PCR

In spite of obtaining product from all the PCR templates tried, cloning was only successful in a proportion. Two independent reactions were obtained for product from KK template and EHEB-1 template; and one from MonoMac6 DNA. One clone was picked from each reaction. Table 4 shows the clones obtained. In summary, there were two type 1 clones (both from the EHEB-1 cell line), two type 2 clones derived from KK DNA, one type 2 clone from MonoMac6 DNA.

TABLE 4
Genomic Clones obtained by PCR Cloning

| Source | PCR-1 | | | PCR-2 |
|---|---|---|---|---|
| Ken K | 2.2 | AN1 | genotype = 2 at | AM1 this clone has not |
| | | | −511 and −31 | been sequenced |
| MonoMac6 | 1.2 | AI3 | genotype −511 = 2, | |
| | | | −31 = 2. | |
| | | AI13 | has not been tested | |
| | | | functionally. | |
| Eheb1 | 1.1 | AJ2 | type 1 | AT1 type 1 |
| Cosmid | 2 | S1 | genotype = 2 | |

### Assessment of Transcriptional Variations Between −511 Type 1 and 2 Promoters

RAW264 cells were transfected with the above constructs and transcriptional activity was determined following addition of various doses of lipopolysaccharide. Two preparations were tried—a commercial preparation, and a highly repurified preparation which was a gift of Dr S. Vogel. Similar results were obtained with both preparations in earlier experiments with pILG-S1 and its mutants, and in these experiments, only the highly repurified preparation was used. Three sets of experiments were performed to investigate transcription of IL1B alleles. All three experiments showed a difference between type 1 and type 2 promoter activities.

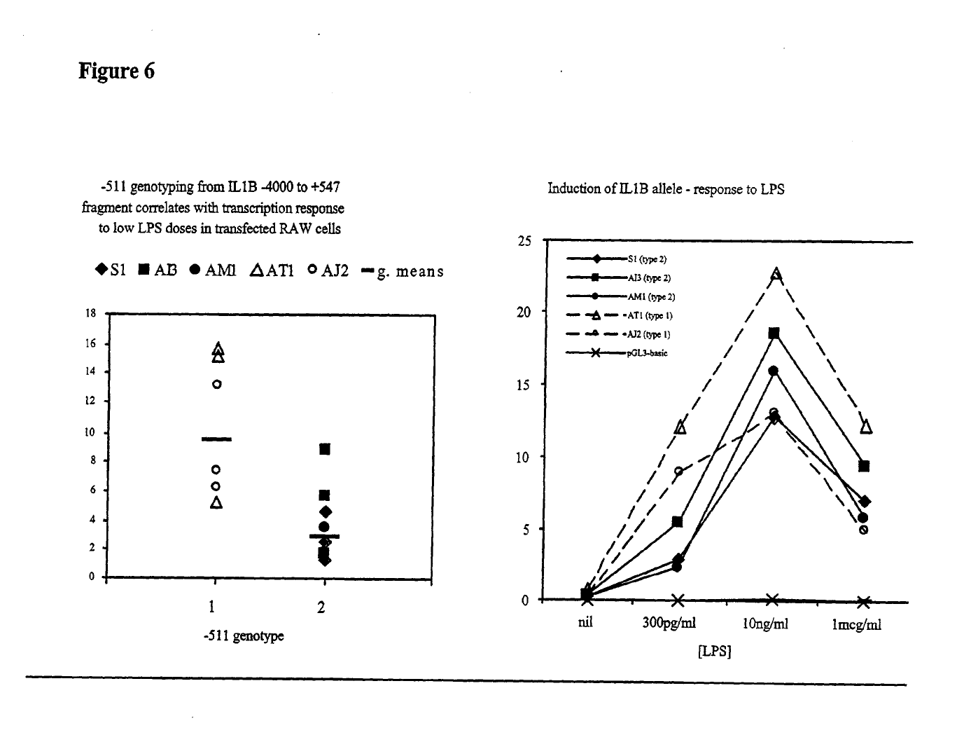

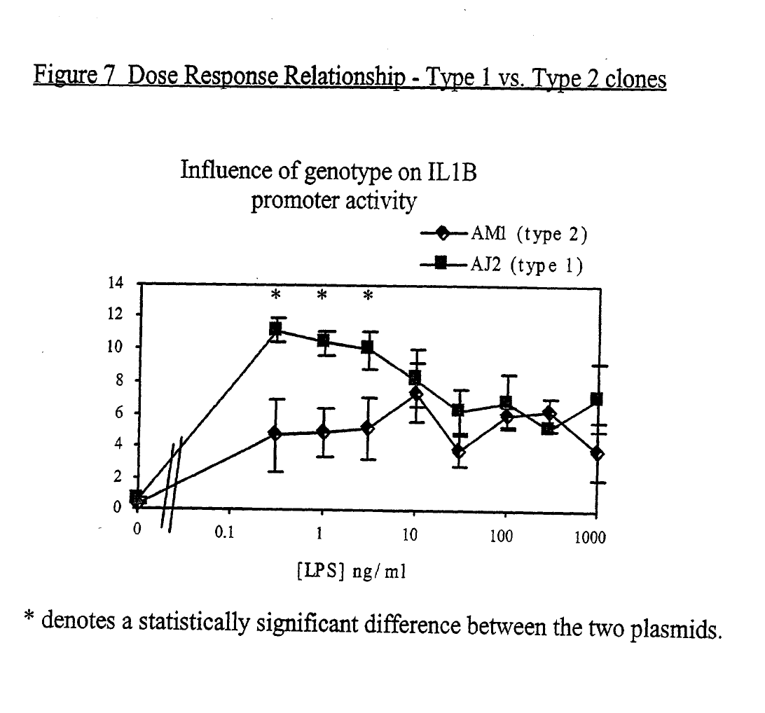

FIG. 6 shows one of the three experiments. Wells were transfected with different alleles. Three wells were transfected with each promoter. The transfections mixtures for each well were set up individually. The left hand panel shows the transcriptional activity of each of the wells when the cells were stimulated with 300 pg/ml of LPS. Increased transcriptional activity is seen with type 1 as compared with type 2 promoters. The difference in the geometric means of type 1 vs. type 2 promoters is significantly different (P<0.01, Kruskall-Wallis). The right panel shows that only at low doses of LPS was this phenomenon evident. This panel shows means of the three triplicate wells transfected at each dose. Error bars are not shown (for clarity) but dev. are ca. 15-20% of the mean at each point. At higher doses (at 6 hrs, the timepoint used in this experiment) the differences apparent at low doses are not evident.

In a second experiment (FIG. 7), the relationship between dose and genotype was tested in more detail. Only clones pILG-AJ2 (type 2, from KK) and pILG-AM1 (type 1, from EHEB-1) were tested (see FIG. 6). The results showed exactly the same pattern as the above experiment. In particular, the plasmids containing one of the novel IL-1B (−3737) polymorphisms showed a 2-3 fold difference in transcription rate between allele 1 and allele 2, with allele 1 being associated with the higher transcription rate. This effect was significant at LPS doses<10 ng/ml. The differential effect on promoter activity was confirmed by specific mutation of the alleles of the novel SNP. Therefore it appears that this novel IL-1B (−3737) polymorphism in the far upstream enhancer region of the IL-1B gene causes a functional difference in transcription in response to LPS.

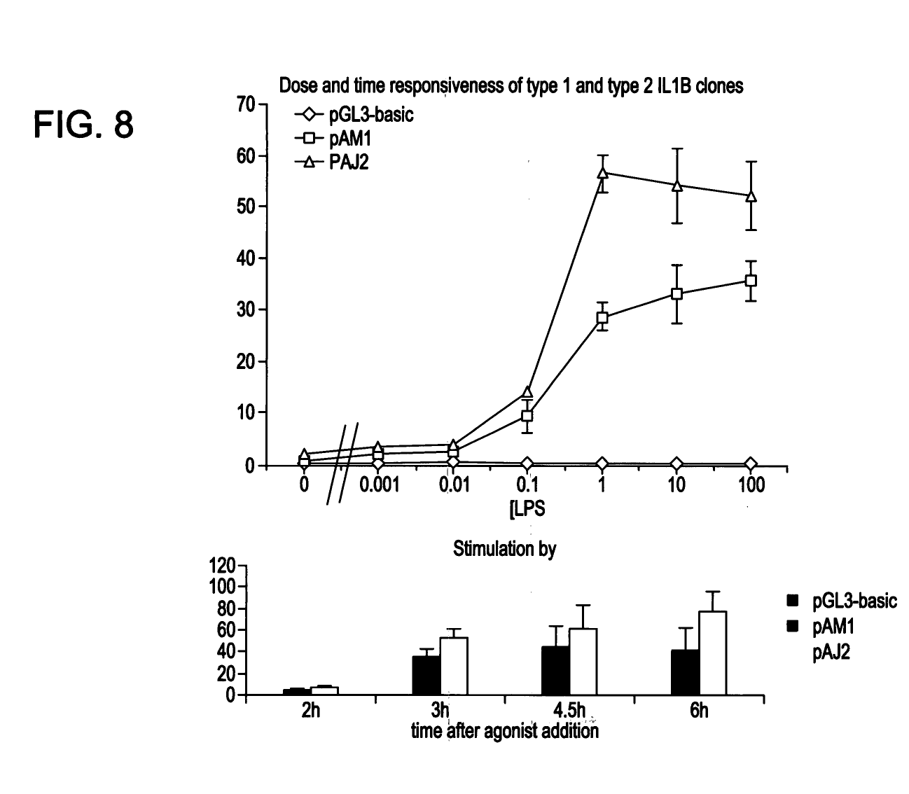

In a third experiment (FIG. 8), the dose response relationship was again tested, as was the relationship between time of assay and the observed difference. In this experiment, there was also a difference between AM1 and AJ2 (type 1 and type 2) clones, but the shape of the dose response curve differed somewhat. The reason for this difference is not clear. All experiments were performed in apparently the same way, but it possible that technical differences, such as the exact cell density may alter cellular behavior.

The lower panel of FIG. 8 shows the influence of sampling time on the differences observed at 6 hours, the time used in all the other experiments. Time was not a crucial determinant of the difference observed. Vehicle was added to control wells in parallel: no reporter induction was observed in these experiments (not shown). In summary, the experiments demonstrate that there are clear and reproducible differences in transcriptional activity (type 1>type 2) demonstrable in all of the experiments performed.

### Sequencing of Clones & Assessment of Functional Potential of New Polymorphisms

In view of the functional differences observed, the genomic clones obtained were sequenced and analysed as described in Methods. Five polymorphisms were detected; two are known, and are the −31 and −511 polymorphisms. Three are novel.

Genome ca. 20 bp up and downstream of these novel polymorphism was compared with the non-redundant human DNA database by BLAST search (http://www.ncbi.nlm.nih.gov/blast). Transcription binding sites were sought in the same fragment used the TRANSFAC 4.0 database using the bioinformatics server at: (http://transfac.gbfbraunschweig.de/TRANSFAC/index.html).

The sequences used are shown below:

For the polymorphism at −3737:

5′ TCTAGACCAGGGAGGAGAATGGAATGT(C/T)CCTTGGACTCTG
CATGT 3′

The sequence shown spans the C/T polymorphism at −3737 of the IL-1B promoter. Allele 1 is C and allele 2 is T.

For the polymorphism at −1469:

5′ACAGAGGCTCACTCCCTTG(C/T)ATAATGCAGAGCGAGCACGAT
ACCTGG3′

The sequence shown spans the C/T polymorphism at −1469 of the IL-1B promoter. Allele 1 is C and allele 2 is T.

For the polymorphism at −999:

5′GATCGTGCCACTgcACTCCAGCCTGGGCGACAG(G/C)GTGAGAC
TCTGTCTC3′

The sequence shown spans the G/C polymorphism at −999 of the IL-1B promoter. Allele 1 is G and allele 2 is C.

The −3737 and −1469 fragments are only found in the human IL-1B gene. The −999 fragment is found in >200 genes, suggesting it is part of a repetitive element. No transcription factor binding sites were identified in the −999 repetitive element, but both the other fragments contain consensus sequences for proinflammatory transcription factors. The −3737 polymorphism is in an NF-kB consensus binding sequence, while −1469 is in an NF-IL6 (C/EBP) consensus binding sequence. In both cases the alignment is with the—strand. The output of the search engine is shown. The codes on the left are links to Transfac entries. The probabilities shown reflect the goodness of match, calculated using two different algorithms, and represent good matches.

−3737 5′ TCTAGACCAGGGAGGAGAATGGAATGT(C/T)CCTTGGACTCTGCATGT 3′
Matrix code start P1 P2
V$NFKB_Q6 | 19(-)| 1.000|0.927|aaGGGAcattccat
−1469 5′ ACAGAGGCTCACTCCCTTG(C/T)ATAATGCAGAGCGAGCACGATACCTGG 3′
Matrix code start P1 P2
V$CEBP_C | 11(-)|0.992|0.901|tgcattatGCAAGggagt
V$CEBPB_01 | 14(-)|1.000|0.967|gcattatGCAAggg

These results are summarized in the table 5 below:

TABLE 5
Polymorphisms detected by this cloning/sequencing project

| SN | Associated with −511 and | Transcription factor binding |
|---|---|---|
| Polymorphism | transcriptional assays | consensus found |
| −31 | Yes | TATA |
| −511 | Yes | None |
| −999 | No | None |
| −1469 | No | C/EBP/NF-IL6 family |
| −3737 | Yes | NF-κB family |

### Conclusions

The previously-unknown −3737 polymorphism lies in a candidate NF-kB binding site in a region of the distal promoter previously shown, by mutagenesis, to be responsible for up to 30% of the activity of the total promoter. Reproducible and significant differences were found when different alleles of this promoter were placed upstream of a reporter gene. Linkage disequilibrium across this region creates haplotypes with the previously known SNPs at −31 and −511 which were shown in these experiments to have no detectable independent effect on transcription of the reporter gene. The results demonstrate that disease associations with these proximal upstream polymorphisms cannot be explained mechanistically by functional alterations caused by these polymorphisms, themselves, and that their linkage to the newly-discovered function-altering polymorphism at −3737 in the distal upstream promoter is the more likely explanation.

### Summary of Experiments

1. RAW264.7 macrophage-like cells respond to fragments of the human I-L1B promoter. A fragment comprising the Asp718I (−4000)→NcoI (+547) fragment was required for maximal responsiveness. This result is in keeping with published data.

2. This region of the human IL1B promoter can be cloned by long distance PCR

3. Two alleles of the IL-1B allele of type 1 (at −511) and three of type 2 (at −511) were obtained from independent PCR reactions, using DNA of caucasian origin as a template.

4. Transcriptional analysis of these clones showed statistically significant differences in transcriptional rate following induction with LPS. These differences were seen in all experiments performed.

5. LPS induction of the IL-1B promoter differed in dose-response relationship from transfection to transfection. The reasons for this were unclear. In some experiments, the difference between type 1 and type 2 alleles was evident at submaximal LPS doses, at which the differences in transcriptional rates between type 1 and type 2 alleles were approximately 2-3 fold.

6. Mutagenesis of a type 2 allele at −31 and −511 did not affect the transcriptional activity of the promoter.

7. The transcriptional differences between type 1 and type 2 promoters must, therefore, be due to polymorphism(s) other than those discovered to date. Automated double stranded sequencing of the clones obtained was performed in order to identify the unknown polymorphisms.

8. Polymorphisms were defined as variations occurring in the same position in different clones. Single base pair changes observed in only one clone were considered to be PCR induced mutations. Five polymorphisms were detected in this stretch of DNA (IL-1B Asp718I (−4000) 4 NcoI (+547). Two of the polymorphisms, at −511 and −31, were already known, the other three have not been described in the literature.

| | Associated | |
|---|---|---|
| Single Nucleotide | with −511 and | Transcription factor binding |
| Polymorphism | transcriptional assays | consensus found |
| −31 | Yes | TATA box |
| −511 | Yes | None |
| −999 | No | None |
| −1469 | No | C/EBP/NF-IL6 family |
| −3737 | Yes | NF-kB family |

9. The previously unknown −3737 polymorphism lies in a candidate NF-kB binding site in a region of the distal promoter previously shown by mutagenesis to be responsible for up to 30% of the activity of the promoter.

5.2. IL-1B (−3737) Polymorphism is Associated with Periodontitis in Chinese Population

While certain IL-1 gene polymorphisms, such as IL-1A (+4845) and IL-1B (+3954), have been associated with severity of periodontal disease in Caucasians, they are found infrequently in some ethnic groups, including Chinese. The novel single-nucleotide polymorphism (SNP), IL-1B (−3737) present in the far upstream enhancer region of the gene for IL-1b, has not previously been studied in this context. Notably, allele 1 of the IL-1B (−3737) polymorphism has been shown to increase transcription rates (see above). In this study we evaluated the population distribution of the IL-1B (−3737) genotype and determined its association with disease in individuals of Chinese heritage.

### Methods

The genotyping for IL-1B (−3737) and other IL-1 SNPs was performed by the TaqMan method. The distribution of IL-1B (−3737) was evaluated in a Caucasian population of 500 adults (age 27-77 years), of unknown periodontal status, and in 300 individuals of Chinese heritage (age 21-69 years). Subjects were considered to be of Chinese heritage if their biological maternal and paternal grandparents and great grandparents were originally from mainland China, Taiwan, Macau, or Hong Kong. To be included in the study, subjects had to be in good general health and have at least 14 natural teeth. The association of IL-1B (−3737) and periodontal disease was determined in the Chinese population by means of multivariate logistic regression models.

### Results

The IL-1B (−3737) genotypes were distributed as shown in the table below:

| IL-1B3 Genotype | Caucasians % (N) | Chinese % (N) |
|---|---|---|
| 1.1 | 30.2 (151) | 22.3 (67) |
| 1.2 | 49.0 (245) | 54.0 (162) |
| 2.2 | 20.8 (104) | 23.7 (71) |

In Caucasians, of the subjects who carried the low transcription genotype, IL-1B (−3737)=2.2 (n=97), 88.3% were also negative for the composite IL-1 genotype (PST®), which includes allele 2 at both IL-1A (+4845) and IL-1B (+3954). Of the subjects who were positive for the composite IL-1 genotype (n=201), 94% carried allele 1, the high transcription allele, at IL-1B (−3737). In the Chinese subjects who were non-smokers (n=163), the IL-1B (−3737) genotype was significantly associated with disease (OR=3.027; 95% CI: 1.139-8.046; p=0.026), with the increased risk being in those carrying the high transcription genotype 1.1.

### Conclusions

In Caucasians, most individuals who were positive for the composite IL-1 genotype were positive for the newly discovered IL-1B (−3737) genotype that increases transcription rate. The IL-1B (−3737) gene polymorphism was found to occur frequently in Chinese, with a similar distribution of genotypes in Chinese and Caucasians. Among Chinese individuals, the IL-1B (−3737) high-transcription genotype was significantly associated with periodontal disease.

5.3. Biacore Binding Analysis of NF-kB binding to the −3737 and Other IL-1 Functional Polymorphism

Kinetic Analysis of the Interaction of p50 Homodimers with DNA

The binding of NF-κB p50 was studied using the BIAcore to obtain kinetic parameters for the interaction of the protein with DNA substrates attached to a streptavidin sensor chip. Duplex 1 contains the consensus NF-κB binding site, duplex 2 and 3 differ by a single nucleotide polymorphism within a consensus sequence (see Table 6). A range of concentrations of the protein were passed over the sensor chip surface, at both low salt conditions (75 mM NaCl) and high salt concentrations (150 mM NaCl). Hart, et al. ((1999) Nucleic Acids Res. 27, 1063-1069).

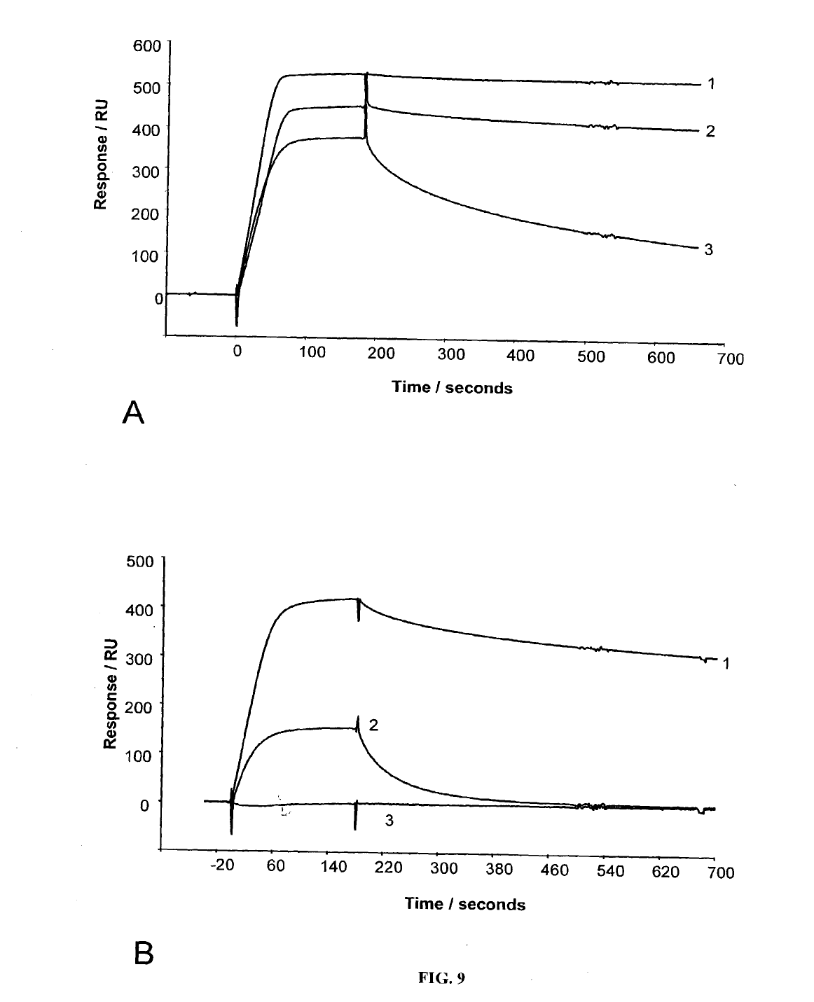

have previously shown that the salt concentration affects both the affinity and specificity of the DNA recognition. Binding of NF-κB p50 to the DNA substrates at low salt concentrations is shown in FIG. 9A. FIG. 9. shows the binding of NF-κB p50 homodimers to DNA substrates. (A) Sensorgram showing the binding of NF-κB p50 (17.5 nM) at 75 mM NaCl to the different duplex DNA substrates. (B) Sensorgram showing the binding of NF-κB p50 (17.5 nM) at 150 mM NaCl to the different duplex DNA substrates. Binding is observed to all 3 of the DNA substrates, however it can be clearly seen from the sensorgram that the dissociation rate constants (the gradient of the dissociation) is different in the 3 complexes. The association and dissociation rate constants were calculated separately from the association and dissociation phases of the sensorgram and are shown in Table 7. These results show that the NF-κB p50 binds to the different DNA substrates with similar association rate constants in the order of 1-5×10⁶ (M⁻¹s⁻¹). However, the dissociation rate constants for the various DNA-protein complexes differ significantly. The NF-κB p50-consensus DNA complex (duplex 1) has the lowest dissociation rate constant (the most stable complex). The equilibrium dissociation constants were calculated using the experimental kₐ and k_{d} values and are shown in Table 7. The NF-κB p50, was shown to bind to its consensus sequence with an affinity of 15 pM (at 75 mM salt), this is in agreement with previous SPR analysis (Hart et al., 1999), whilst the affinity of duplex 2 was 130 pM and duplex 3, 2000 pM.

The SPR analysis was then repeated at a higher salt concentration, resembling more physiological conditions (0.15M NaCl). The binding of NF-κB p50 to the DNA substrates is shown in FIG. 9B. The results show that no binding is seen to duplex 3 under these conditions. Again duplex 1 and 2 show similar association but different dissociation kinetics, also the level of protein binding to duplex 2 (as seen by the level of response) is much lower compared to the binding at 75 mM NaCl. The kinetic data for the binding at 150 mM NaCl are shown in table 7. The results again show similar association rate constants, but significantly different dissociation rate constants. The dissociation rate constants for both protein-DNA complexes are higher compared to the rates at 75 mM NaCl, indicating decreased stability of the complexes at the higher salt concentration. Moreover, there is now a 36 fold difference in the dissociation rate constants of duplex1/2—NF-κB p50 complexes, compared to the 4 fold difference seen at the lower salt concentration. The equilibrium dissociation constants for the consensus sequence—NF-κB p50 binding is 0.2 nM and 12 nM respectively indicating a 60 fold difference in affinity compared to the 9 fold difference in affinity seen under low salt conditions These results show that at the higher salt concentration the overall affinity towards the DNA substrates is reduced, however the specificity of the DNA recognition is increased, with no binding seen to duplex 3 and a 60 fold difference in affinity comparing the consensus sequence with duplex 2.

### Molecular Recognition of the IF-KB Binding Site

Two crystal structures have been obtained for the interaction of NF-κB p50 homodimers bound to DNA substrates (see, Müller, et al. (1995) Nature, 373, 311-317; Ghosh, et al. (1995) Nature, 373, 303-310). Although the two co-crystal structures contained DNA substrates of different length and sequence, there are many similarities. In each p50 homodimer subunit two Arg side chains donate a pair of hydrogen bonds to the two central guanines (G₂ and G₃ see table 6). These contacts are predicted to be the most critical components of DNA recognition (Müller et al., 1995). A Lys residue is also shown to make specific contacts to the innermost G₄, although the specificity is less predictable due to the relatively unconstrained nature of the Lys side chain. The outermost G₁ was also identified in the structure from Müller et al., 1995 to make contacts with a His side chain. There are many other specific interactions between the side chains and bases, which differ slightly in the two co crystal structures. This suggests that some of the DNA binding elements are flexible and therefore enable the recognition of different sequences within the variable portion of the consensus sequence. The effects of a SNP on the DNA recognition of p50 homodimers was examined by comparing the kinetic data obtained using duplexes 2 and 3 in the SPR experiments. Duplex 3 contains an A/T base pair at the +4 position compared with the G/C base pair in duplex 2. The G₄ is shown to make important interactions to Lys (241 numbered from Ghosh et al., 1995) in the in the crystal structure (see FIG. 9B). However, replacement of the guanine with adenine abolishes this interaction and presents a possible steric clash between the Lys side chain and the N6 amino group of adenine. The effect of this interaction is clearly demonstrated in the affinity data presented here, which shows under low salt conditions a 15 fold reduction in affinity. Under higher salt concentration this difference is expected to be much larger, as no binding is seen to the duplex 3 due to the very fast off rate and instability of the protein—DNA complex. These results demonstrate the dramatic effect of the SNP on the molecular recognition of NF-κB p50. The results also show the effect of alterations at the G₁ position in the consensus sequence. In comparison to duplex 1 (consensus sequence), duplex 2 contains an A/T base pair at positions 1 and 12. The crystal structure (Müller et al., 1995) shows that His 67 makes contacts to the G₁ in each p50 subunit (see FIG. 9D) replacement of the G₁ with adenine again abolishes this interaction. Again this effect is witnessed in the affinity data. Under low salt conditions there is a 9 fold reduction in affinity, and under high salt this is increased to a 60 fold difference. In conclusion the affinity data presented here shows the alteration of the G₄ to an A₄ causes a much larger effect on the affinity of the DNA interaction of NF-κB p50 compared with the G₁ to A₁ alteration. These effects on the affinity can be readily reconciled with the structural data.

### Materials and Methods

### Oligonucleotide Substrates

Oligonucleotide synthesis was performed on an Applied Biosystems 394 DNA synthesiser using cyanoethyl phosphoramidite chemistry. The biotin phosphoramidite was obtained from Glen Research. Three duplex DNA substrates were generated by annealing complementary oligonucleotides of 23 bases in length in which one the strands was biotinylated at the 5′-end. Annealing was performed at a final DNA concentration of 1 μM in 10 mM Tris-HCl (pH. 7.4), 0.1M NaCl, 3 mM EDTA by heating to 95° C. for 5 minutes and cooling to 25° C. over 35 minutes. The sequences used in the construction of the duplex DNA were: Duplex 1 5′-biotin-AGTTGAGGGGACTTTCCCAGGC and the complementary 5′-GCCTGGGAAAGTCCCCTCAACT. Duplex 2, 5′-biotin-GAGAATGGAATGTCCCTTGGACT and the complementary 5′-AGTCCAAGGGACATTCCATTCTC. Duplex 3, 5′-biotin-GAGAATGGAATGTTCCTTGGACT and the complementary 5′-AGTCCAAGGAACATTCCATTCTC. The underlined region is the p50 binding site, the bold letters indicate the SNP analysed in this study.

### Surface Plasmon Resonance

Surface plasmon resonance (SPR) was performed using a BIAcore 2000™ (Uppsala, Sweden). Oligonucleotides were diluted in HBS buffer (10 mM HEPES pH 7.4, 75-150 mM NaCl, 3 mM EDTA, 0.05% (v/v) surfactant P20) to a final concentration of 1 ng/ml and passed over a streptavidin sensor chip (SA) at a flow rate of 10 μl/min until approximately 50 response units (RU) of the oligonucleotide was bound to the sensor chip surface. The recombinant (human) NF-κB p50 (Promega) was also diluted in HBS buffer containing either 150 mM or 75 mM NaCl and a range of concentrations (2-100 nM) were injected over the DNA-charged sensor chip at a flow rate of 20 μl/minute for 3 min and allowed to dissociate for 5 min. Bound protein was removed by injecting 10 μl of 1M NaCl. This regeneration procedure did not alter the ability of NF-κB p50 to the DNA. Analysis of the data was performed using BIAevaluation software. To remove the effects of the bulk refractive index change at the beginning and end of injections (which occur as a result of a difference in the composition of the running buffer and the injected protein), a control sensorgram obtained over the streptavidin surface was subtracted from each protein injection. All assays were performed at 25° C.

### Kinetic Analysis

The rate of complex formation in a binary association is described by:

dR/dt=kₐC(Rₘₐₓ−R)−k_{d}R (1)

where dR/dt is the rate of change of the SPR signal, C is the concentration of analyte, Rₘₐₓ is the maximum analyte binding capacity in RU and R is the SPR signal in RU at time t. The equation can be rearranged to give:

dR/dt=kₐCRₘₐₓ−(kₐC+k_{d})R

Sensorgrams were recorded at a minimum of five different analyte concentrations and dR/dT against R was plotted for each concentration. The gradient of each of these lines (kₐC+k_{d}) represents the observed association rate, −k_{obs}. A plot of −k_{obs} against C allows kₐ to be determined from the equation below.

−k_{obs}=kₐC+k_{d}

At the end of the sample injection the protein was replaced by running buffer and the bound protein was dissociated from the DNA. Since the concentration of protein in the running buffer is zero and assuming the rebinding was negligible then the dissociation rate constants can be calculated using linear regression analysis assuming a zero order dissociation using the following equation:

dR/dt=−k_{d}R₀e^{-kd(t-t)}₀

Where dR/dt is the rate of change of the SPR signal, R and Rₒ, is the response at time t and t_{o.} k_{d} is the dissociation rate constant.

The equilibrium dissociation constant (K_{D}) can be obtained from the ratio of the rate constants:

K_{D}=k_{d}/kₐ

| DNA | Sequence 5′-3′ |
|---|---|
| Duplex 1 | AGTTGAGGGGACTTTCCCAGGC |
| | TCAACTCCCCTGAAAGGGTCCG |
| Duplex 2 | AGTCCAAGGGACATTCCATTCTC |
| | TCAGGTTCCCTGTAAGGTAAGAG |
| Duplex 3 | AGTCCAAGGAACATTCCATTCTC |
| | TCAGGTTCCTTGTAAGGTAAGAG |
| Consensus | 1 2 3 4 5 6 7 8 9 10 11 12 |
| | GGGGXNYYYCCC |
| | CCCCYNXXXGGG |

TABLE 6
Oligodeoxynucletide substrates used in the SPR binding analysis. The consensus
sequence and numbering scheme is shown below, where X indicates a purine and Y a
pyrimidine.

| | |
|---|---|
| NaCl | Rate constant |

| (mM) | DNA | kₐ (M⁻¹s⁻¹) ± S.D. | K_{d} (s⁻¹) ± S.D. | K_{D} (M) ± S.D. |
|---|---|---|---|---|
| 75 | 1 | 4.0 × 10⁶ ± 0.9 × 10⁶ | 5.81 × 10⁻⁵ ± 0.8 × 10⁻⁶ | 1.5 × 10⁻¹¹ ± 0.5 × 10⁻¹¹ |
| 75 | 2 | 3.2 × 10⁶ ± 0.9 × 10⁶ | 2.5 × 10⁻⁴ ± 0.2 × 10⁻⁴ | 1.31 × 0⁻¹⁰ ± 0.8 × 10⁻¹⁰ |
| 75 | 3 | 1.5 × 10⁶ ± 1.0 × 10⁶ | 2.1 × 10⁻³ ± 0.7 × 10⁻⁴ | 2.0 × 10⁻⁹ ± 0.7 × 10⁻⁹ |
| 150 | 1 | 2.7 × 10⁶ ± 0.9 × 10⁶ | 5.0 × 10⁻⁴ ± 0.1 × 10⁻⁴ | 2.0 × 10⁻¹⁰ ± 0.5 × 10⁻¹⁰ |
| 150 | 2 | 1.6 × 10⁶ ± 0.4 × 10⁶ | 1.8 × 10⁻² ± 0.1 × 10⁻⁴ | 1.2 × 10⁻⁸ ± 0.9 × 10⁶ |

| | | |
|---|---|---|
| 150 | 3 | No binding |

Table 7. Kinetic rate constants (kₐ and k_{d}) and calculated equilibrium binding constants (K_{D}) for the binding of p50 to the oligodeoxynucleotide substrates.

### 5.4. Discovery of Additional Functional Polymorphisms

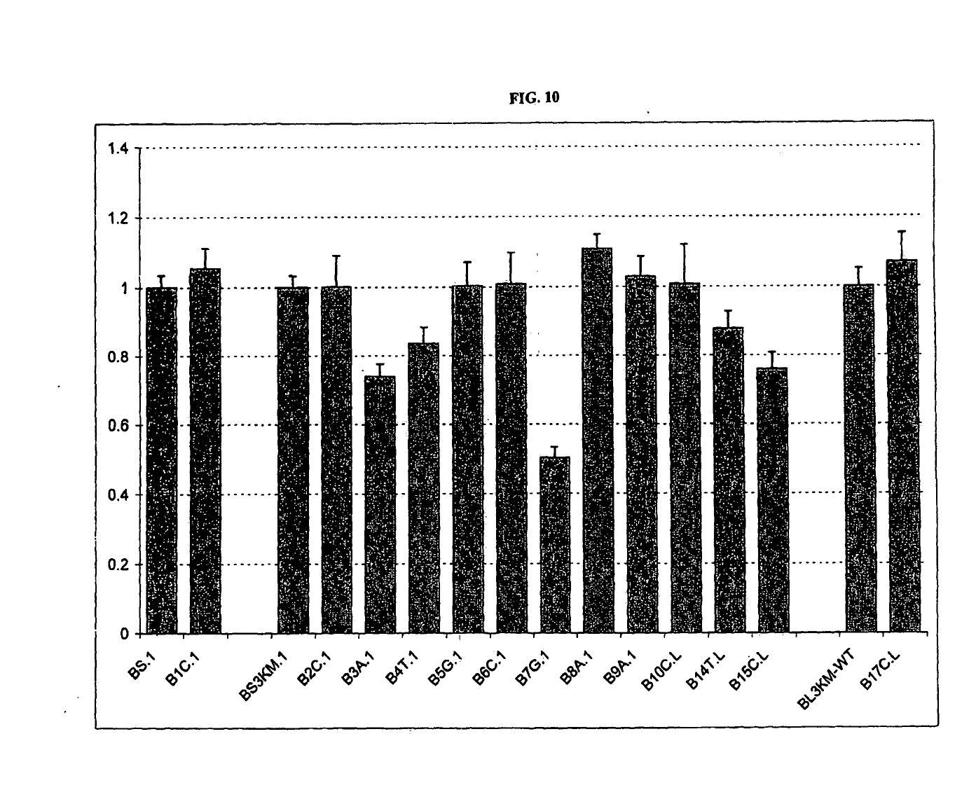

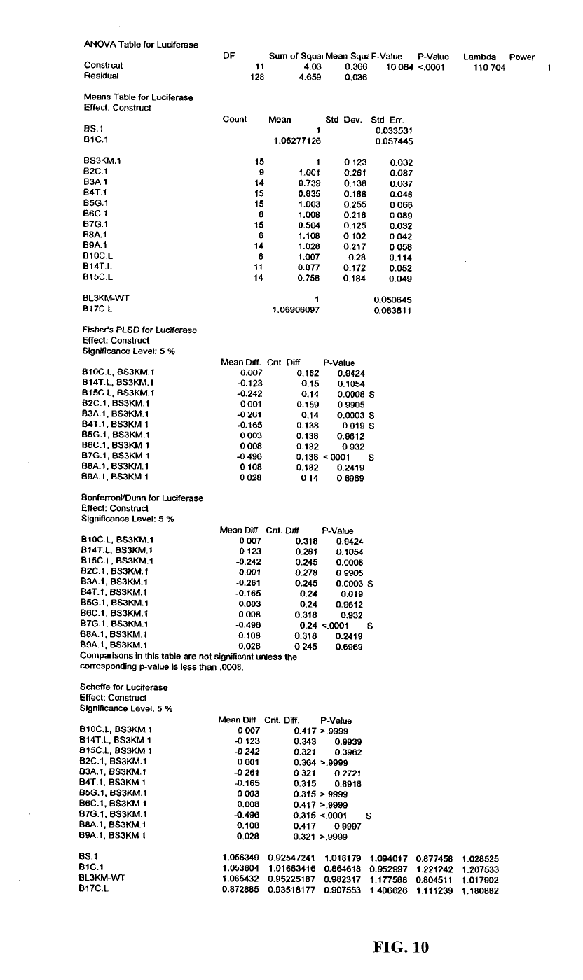

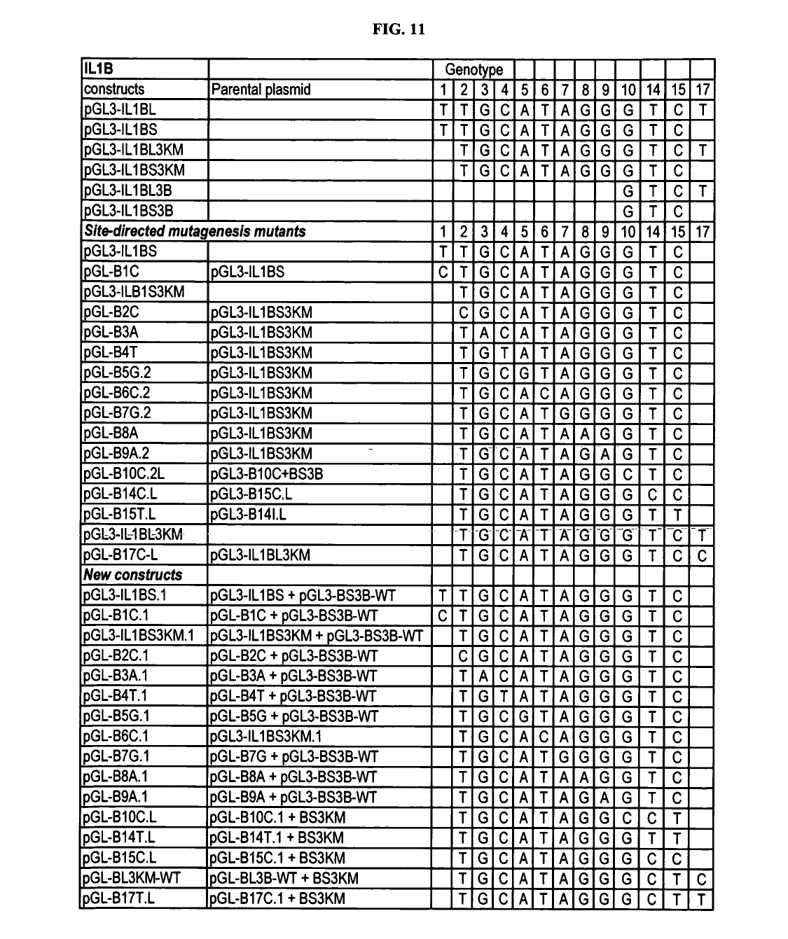

The genetics discovery group has confirmed that the IL-1B4 SNP (−3737) is functional by transfection analysis in RAW cells (see FIG. 10) and, in addition, found other polymorphisms that arc also functional in this assay as follows. The strategy of the constructions and sequence information for the functional SNP analyses is shown in FIG. 11 which indicates the names of all the constructs created and analyzed.

Three additional functional SNPs, called IL-1B3, IL-1B7 and IL-1B15, were identified (these SNP names utilize the nomenclature system for the individual allele2 polymorphisms shown in FIG. 11).

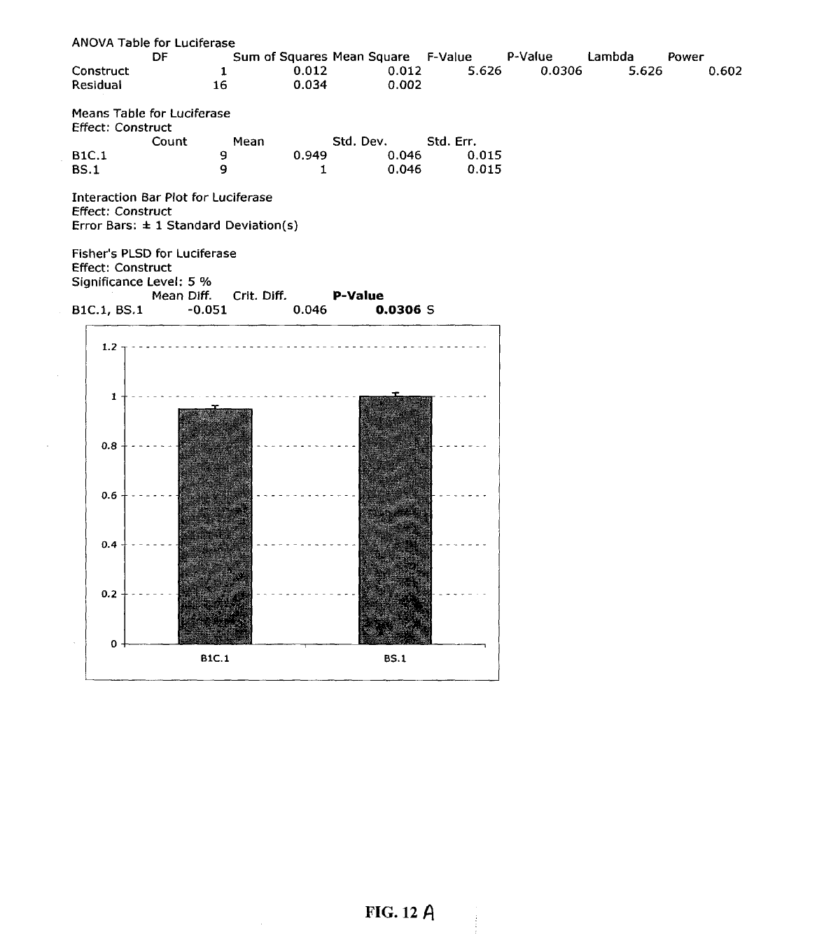

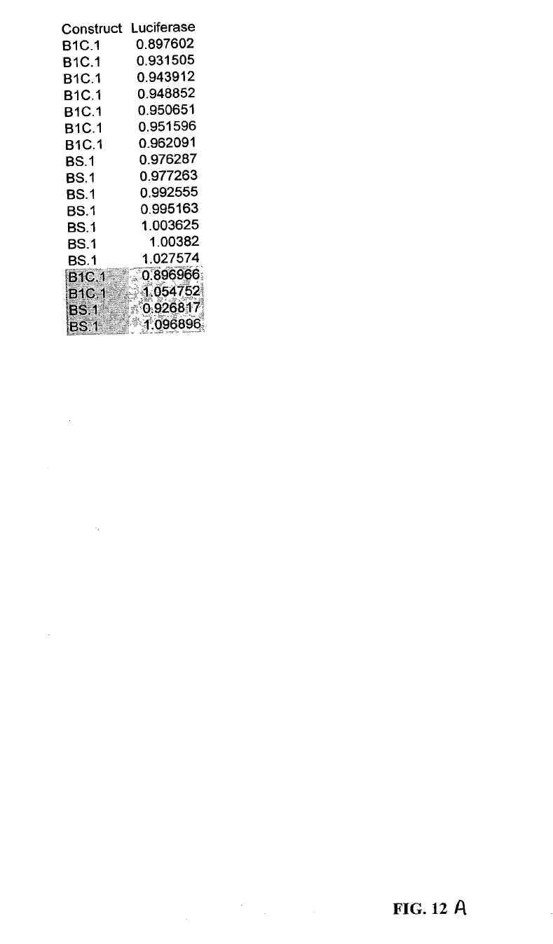

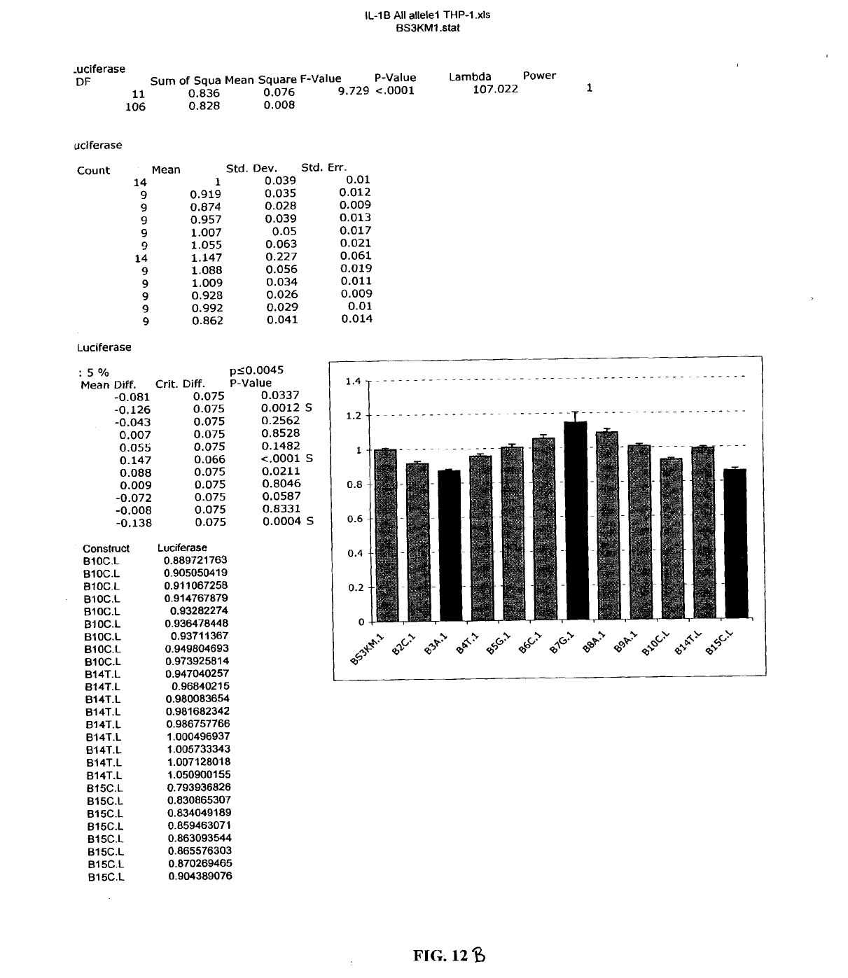

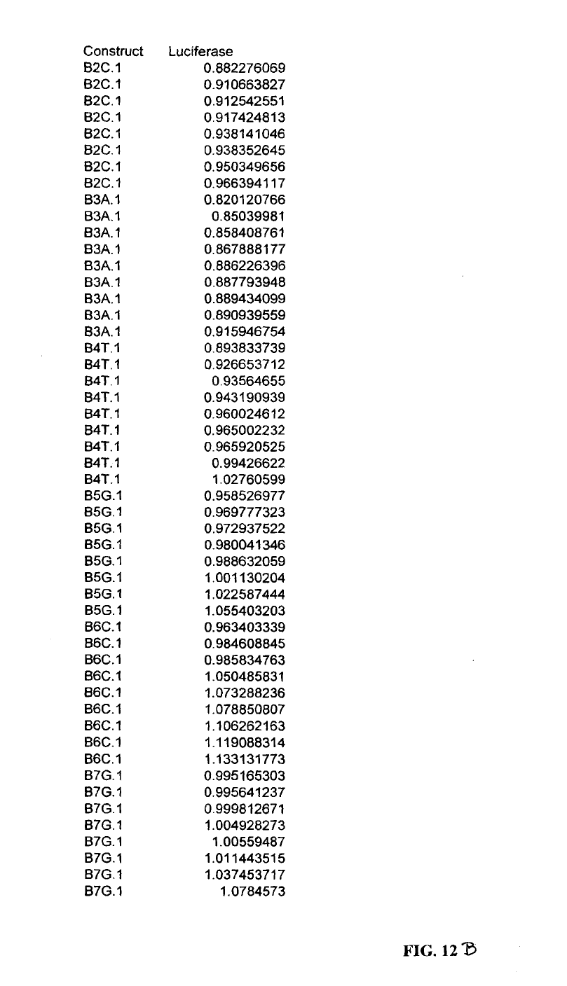

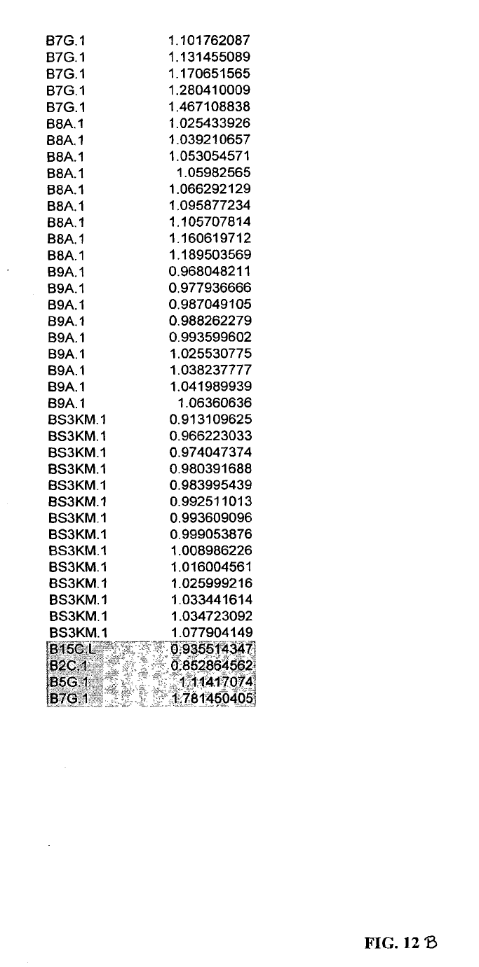

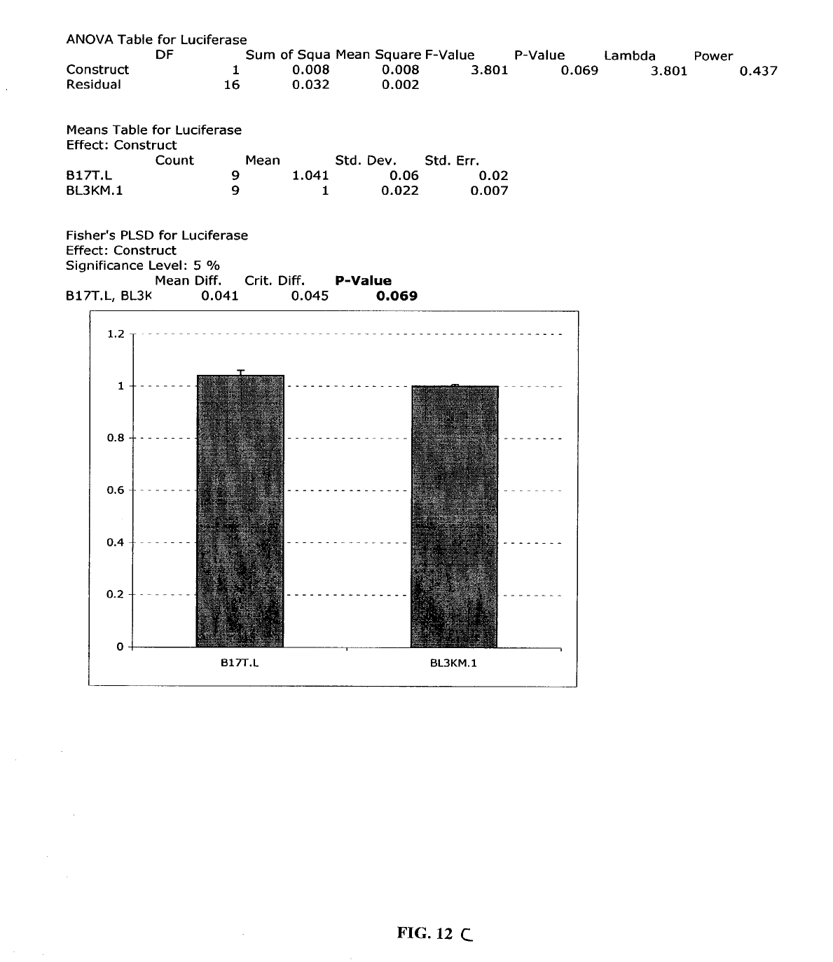

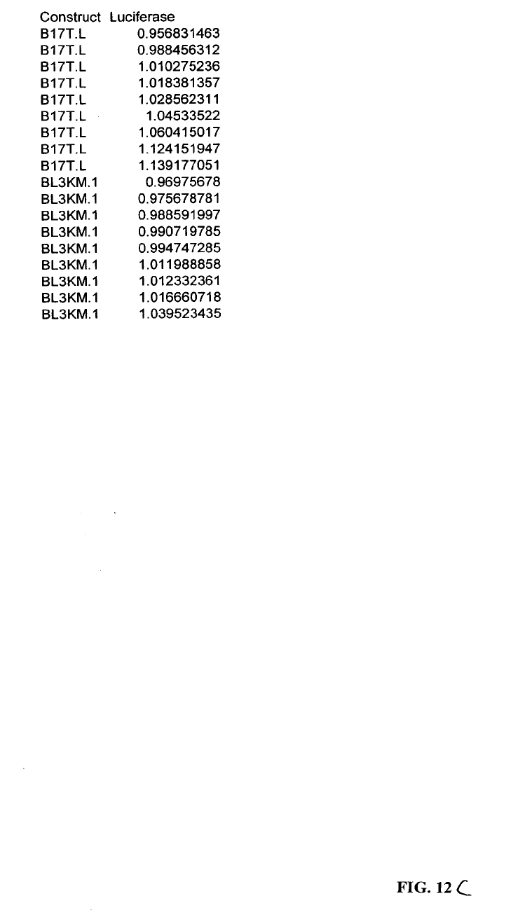

IL-1B3 allele 2 and IL-1B15 allele-2 reduce the rate of transcription in RAW (murine macrophage cells) and in THP-1 cells (human monocyte cells) (see sequence data in FIG. 11 and experimental data in FIGS. 10 and 12). IL-1B7 allele-2 (genotype TGCATGGGGTC) reduces transcription rate in RAW cells (see FIG. 10) IL-1B7 allele-2 increases transcription rate in THP-1 cells (see FIG. 12) (allele 1 SNPs). FIG. 10 also shows that IL-1B3 (genotype TACATAGGGTC) and IL-1B15 (genotype TGCATAGGGTT) significantly decrease expression of IL-1B in RAW cells.

### INCORPORATION BY REFERENCE

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1.-14. (canceled)

15. A method of diagnosing an increased likelihood of developing an inflammatory disease or condition associated with increased interleukin production in a human subject comprising:
obtaining a sample of nucleic acid from a human subject; and
determining the identity of the −3737 IL-1B allele as a type 1 or a type 2 promoter sequence,
wherein the presence of a type 1 IL-1B promoter sequence is diagnostic of an increased likelihood of developing an inflammatory disease or condition associated with increased interleukin production.

16. The method of claim 15, wherein the inflammatory disease is periodontal disease.

17. The method of claim 15, wherein the inflammatory disease is Alzheimer's disease.

18. The method of claim 15, wherein the inflammatory disease is selected from the group consisting of: Alzheimer's Disease, Amylotropic Lateral Sclerosis, arthritis, collagen-induced arthritis, juvenile chronic arthritis, juvenile rheumatoid arthritis, osteoarthritis, asthma, cardiovascular diseases, autoimmune diabetes, insulin-dependent (Type 1) diabetes, diabetic periodontitis, diabetic retinopathy, diabetic nephropathy, celiac disease, chronic colitis, Crohn's disease, inflammatory bowel disease, ulcerative colitis, gastric ulcers, hepatic inflammations, cholesterol gallstones, hepatic fibrosis, Kawasaki's Syndrome, multiple sclerosis, nephropathies, neurodegenerative disease, ophthalmopathies, pancreatic acinitis, periodontal disease, pulmonary diseases, restenosis, rheumatoid arthritis, thyroiditis, alopecia aerata, autoimmune myocarditis, and Graves' disease.

19. (canceled)

20. A method of predicting an increased likelihood of developing an inflammatory disease or condition associated with increased interleukin production in a human subject comprising: obtaining a sample of nucleic acid from the human subject; and detecting the presence of an IL-1 haplotype associated with a −3737 IL-1B type 1 allele, wherein the presence of the IL-1 haplotype associated with the −3737 IL-1B type 1 allele is diagnostic of an increased likelihood of developing the inflammatory disease or condition.

21.-23. (canceled)


SEQUENCE LISTING (78 sequences)

SEQ ID NO: 1 - DNA, 45 bp, Homo sapiens
tctagaccagggaggagaatggaatgtcccttggactctgcatgt

SEQ ID NO: 2 - DNA, 45 bp, Homo sapiens
tctagaccagggaggagaatggaatgttccttggactctgcatgt

SEQ ID NO: 3 - DNA, 47 bp, Homo sapiens
acagaggctcactcccttgcataatgcagagcgagcacgatacctgg

SEQ ID NO: 4 - DNA, 47 bp, Homo sapiens
acagaggctcactcccttgtataatgcagagcgagcacgatacctgg

SEQ ID NO: 5 - DNA, 49 bp, Homo sapiens
gatcgtgccactgcactccagcctgggcgacagggtgagactctgtctc

SEQ ID NO: 6 - DNA, 49 bp, Homo sapiens
gatcgtgccactgcactccagcctgggcgacagcgtgagactctgtctc

SEQ ID NO: 7 - DNA, 11 bp, Homo sapiens
tgcatagggtc

SEQ ID NO: 8 - DNA, 11 bp, Homo sapiens
tgcatagggtc

SEQ ID NO: 9 - DNA, 11 bp, Homo sapiens
tgcatagggtc

SEQ ID NO: 10 - DNA, 11 bp, Homo sapiens
tgcatagggtc

SEQ ID NO: 11 - DNA, 11 bp, Homo sapiens
tgtatagggtc

SEQ ID NO: 12 - DNA, 11 bp, Homo sapiens
tacatagggtc

SEQ ID NO: 13 - DNA, 11 bp, Homo sapiens
tgcatggggtc

SEQ ID NO: 14 - DNA, 11 bp, Homo sapiens
tgcatagggtt

SEQ ID NO: 15 - DNA, 11 bp, Homo sapiens
tgcatagggtc

SEQ ID NO: 16 - DNA, 11 bp, Homo sapiens
tgcatagggtc

SEQ ID NO: 17 - DNA, 11 bp, Homo sapiens
tgcatagggtc

SEQ ID NO: 18 - DNA, 11 bp, Homo sapiens
tgcatagggtc

SEQ ID NO: 19 - DNA, 11 bp, Homo sapiens
tgtatagggtc

SEQ ID NO: 20 - DNA, 11 bp, Homo sapiens
tacatagggtc

SEQ ID NO: 21 - DNA, 11 bp, Homo sapiens
tgcatggggtc

SEQ ID NO: 22 - DNA, 11 bp, Homo sapiens
tgcatagggtt

SEQ ID NO: 23 - DNA, 24 bp, Artificial Sequence
  Description of Artificial Sequence Primer
tgtacctaagcccaccctttagag

SEQ ID NO: 24 - DNA, 20 bp, Artificial Sequence
  Description of Artificial Sequence Primer
tggcctccagaaacctccaa

SEQ ID NO: 25 - DNA, 20 bp, Artificial Sequence
  Description of Artificial Sequence Primer
gctgatattctggtgggaaa

SEQ ID NO: 26 - DNA, 20 bp, Artificial Sequence
  Description of Artificial Sequence Primer
ggcaagagcaaaactctgtc

SEQ ID NO: 27 - DNA, 45 bp, Homo sapiens
tctagaccagggaggagaatggaatgtyccttggactctgcatgt

SEQ ID NO: 28 - DNA, 47 bp, Homo sapiens
acagaggctcactcccttgyataatgcagagcgagcacgatacctgg

SEQ ID NO: 29 - DNA, 49 bp, Homo sapiens
gatcgtgccactgcactccagcctgggcgacagsgtgagactctgtctc

SEQ ID NO: 30 - DNA, 45 bp, Homo sapiens; identical to SEQ ID NO: 27

SEQ ID NO: 31 - DNA, 14 bp, Homo sapiens
aagggacattccat

SEQ ID NO: 32 - DNA, 47 bp, Homo sapiens; identical to SEQ ID NO: 28

SEQ ID NO: 33 - DNA, 18 bp, Homo sapiens
tgcattatgcaagggagt

SEQ ID NO: 34 - DNA, 14 bp, Homo sapiens
gcattatgcaaggg

SEQ ID NO: 35 - DNA, 22 bp, Artificial Sequence
  Description of Artificial Sequence Synthetic oligonucleotide
agttgaggggactttcccaggc

SEQ ID NO: 36 - DNA, 22 bp, Artificial Sequence
  Description of Artificial Sequence Synthetic oligonucleotide
gcctgggaaagtcccctcaact

SEQ ID NO: 37 - DNA, 23 bp, Artificial Sequence
  Description of Artificial Sequence Synthetic oligonucleotide
gagaatggaatgtcccttggact

SEQ ID NO: 38 - DNA, 23 bp, Artificial Sequence
  Description of Artificial Sequence Synthetic oligonucleotide
agtccaagggacattccattctc

SEQ ID NO: 39 - DNA, 23 bp, Artificial Sequence
  Description of Artificial Sequence Synthetic oligonucleotide
gagaatggaatgttccttggact

SEQ ID NO: 40 - DNA, 23 bp, Artificial Sequence
  Description of Artificial Sequence Synthetic oligonucleotide
agtccaaggaacattccattctc

SEQ ID NO: 41 - DNA, 15402 bp, Homo sapiens
  modified_base (5816)..(5817): a, t, g, c, other or unknown
gcaaaagtaatcacggtttttgtcattaaaagttttgccattacttttaatgataaaaaccacgattacttttgcgccaacttaatagctcactgcagcctcaaaattcctggtctcagggaatcctcctgcctcagcttcctgaatagctgggactacaggcacatgcaatcctacctggctaattttttaaaaattttttttgtaaagatagaaactcattttgttgtccaggctggtttcaaactcttgtctttgtgcctccctctgccctgtgcaagaccttctggatgcccactaatgaagacttccagggagaggaaaagtaaacataggtccctgatcaagggaccagggtttatcgaccacaaacagcatgcccagattccactggcagtcctagaggtcgcatttgccccaagtgtgtgtggaaggcctctccctagcagttggtttatacaccagccacagcacagcatattctcttaaattgtgaacatttgcaaaacctccttgaggacaactatcatgtcttgtgtacttttgtttcccttcccctatgtacacgcacgcgcgcgcacacacacacacacacacacccctcaaactgaatgcctggtgtgctgaatggatgaatggctaatgtaagtcattctaaaagctactttctttggcataccatcacctttgatttcatctttctggaactcctatgttcccagatgaatttggaaagccctcaggaaacatttcaaaattgctatatgggagaaatgggagggtctctctagaaatttacctgccacaggtatttctggtaagacacagcaaaggtggcaccacccattcctcgttacaatgtcaatgccagtcaccttcctgtcccataaaactttattaaaggtgcagaattcccatggaagcaggtggacaccatctgcttccagccagccaggggagcaaggtgtccactgtgcctttgtggcaggaactgcgcttctctactctcccactttgaggcctctggggctggcctgctgcctcctcattgacaaggctgcttactgagcagttcattctgagctggacatagtgcttctggtgagtctctacttctatttaacccaaagatattctttcctaaggaaacgctttcctgtcgggggaggttagctccagatggaagtcacaagtgatggcatggtagctctcatccgtttgggtggatgatattcacggagcaccaccatgagccagtcatggaggtgaacagtatatgccagccctgaatcaggtgcattgacagcaagggagacaagcaaacaaagctgaggtttgctgaggatgttcaagactcacacagcacagaggagcatccaccacccagcttgggaaaggacttgttatagagggggtgaagcatgagctgagtcttgaaagactagaaattagccaaactacaaggaggagaaggagtttccagtcaggaagaacaggttatgcaaaagcacagagactagaaagaatatcacattcaaggaactgcaaatagacaggaaaggttgatgcgtgggataggagaggagggcaggggattccaggtgggccctgcttgccacactcaggagcttgaacttatccacaaaggagatgtggaaccagtaatgaatgggttttgtgcaagggcttcatgtcaccagatttgctttttggagatacttctgtggctgatatgtgaggaagggatggaggaagtttccgtggcaatcaggaaaaccaattagcagatgattcaaatggcctaggggaaaagggaggaggacttggactaccatgcagcagcagaaatggagagaaataacagatcccaggcactcaggaagcgctcagaatgagcccttcaaagaacttatggtaggtgatggatggatggagtgtgagtcctgggatagcattgcctgggaaaatactttctagttgagacagggaagtgggccagcagaaatggagggcttcttctttttgctttaaatacttttataatatttggaactttgaaaatgagcagatatattagcaaaaagcctaaaagggatatttttgaaatcactgctagttctaacatataactttcagcttgcacacatcatcaattaactttgatagcgcctttctgaaactatcatcccaaatagcaatccttgtaaaaacctattttgaaaaacgggccttgtaggatagcctcacagatgttttgtggtagattttctaacattctaatgtcagggagtgaaaggaatcccgttagaagttggaaaattctggaatctctattcatggtattaaagttttgccgtcacacaaaagtttaacacctttacacaatcagacttcctcattttacattgctcggtaattagaggaaatcagtcacccagagcctgggtcctagacttgacaaaatgcacccaacaaatcctgagtggccttgctgaggacttctcccagaagatagaaaactcagttccagccaacaagggggaagcagctgaagaagtgaaattaacaaagtcctggaaggaaatgaccaaatcatctttgattgtgtaataaccagagagtagaatacagcaacgacagacattttgggagagaagcattttatcatagcttttagaagagaagtatttttcagcatcataagcacacaattccaaggacagataccttcaagggattgcttttgacagttatgacaaagtcttaaagaagaataaaaggacaaaggaaatcctccagcaacaaagctgccacttatagatgagaaagtgaatgggaataaggaagaaactcagaaaagggaagagagatcactaaaaaccctgatttggaaagtcccagtactaccctgagaggagaaagaaacaaattcacacagcacgtcaccgccagaagaagaaaggagggaagacaagggaacagagaggatctcaatcctaaaaggacaatgtggaaacatttaggggacagaggtgaatctgccaggccaatgtagtttagaatgtcactcaatcactgagaatgagaaaggagtctgccgatgggcaccatgtggacaggagatgaggcaataaacatatgtaacaattaaaagtgagaaataaagatctggttttggttcagttaacccatatttgagtcgtagttccagcatttactgtttgtgcggtcttaaataatttattgtctcaacctcagtttctgcataggtcaaatggttcaatattatctactttaaaggttattatgaagagttaataagataatgagggaaaaaaaggtacctggcacttagtaggtgctcaataaaggacggctttttttttttaagtattgcttctaaatttgtatgtaagaaaaaatgatataatacaatgatataagacaggggaccgcaggacaagtccagccacacccattcatttatgtattatctgactgcttttctgaaatgatgcaaggttgagtagttgtacctgaaacatttattatctggccctttacagaaaatgtttccagaccctggataagtggtaccagagccccctctgtttgtggtcccctctcttatacccactaggtgtgagaaaagacatagagtaggagagccctgccatccatcttacccacccaggggctttttctgatggatccaaaggaaggacaaggtcttattggtctcccagaactgacataacaactccgacatcagggaaaagccattggagactacatagctcgccagccccagccacctgctcatatatctaagccctccttgttctagaccagggaggagaatggaatgtcccttggactctgcatgtccccaatctgagaacctggatccaagagggagaagaagcccattggagatgatgccataaaggaagtggaagcgatatgataaaaatcatagtgcccattcccaaataatcccagaagcagaagggaaaggagagaaatatccacaaagacaggtgtgggtacacacaacatttttcatactttaagatcccagagggactcatggaaatgatacaagaaaatgactcataagaacaaatattaggaagccagtgccaagaatgagatgggaaattggggaaaatgttgggggcagattgcttagttctgttctaagcaagagggtgaacaaggaaggaacagctcactacaaagaacagacatcactgcatgtacacacaataatataagaactaacccatgattattttgcttgtcttcttgttcaaaatgattgaagaccaatgagatgagatcaaccttgataactggctgcgaagcccatgattagacacaagatggtatcagggcacttgctgctttgaataaatgtcagtctcctgtcttggaagaatgacctgacagggtaaagaggaacttgcagctgagaaaggctttagtgactcaagagctgaataattccccaaaagctggagcatcctggcatttccagctccccatctctgcttgttccacttccttggggctacatcaccatctacatcatcatcactcttccactccctcccttagtgccaactatgtttatagcgagatattttctgctcattggggatcggaaggaagtgctgtggcctgagcggtctccttgggaagacaggatctgatacatacgttgcacaacctatttgacataagaggtttcacttcctgagatggatgggatggtagcagatttgggtccaggttacagggccaggatgagacatggcagaactgtggagactgttacgtcagggggcattgccccatggctccaaaatttccctcgagcgaaagcatcaggggctcatgcaacctggatactagtgctgcttcaaccacactgtgctattggatgagtcacttccaccctcctagccttgatttcttcgtctgctgttcacattcaaatagctattcatgtcttcatctctgtggtcccaccatatcccaccagacaatcattagggctcctcttagctggcagattctgaggtcctggatgctacaattggaagatggagaagtagaagctcaaggtttctgacctgtatcccaagtcccagaagcagaatggactaactcagagctgatgctcgggtcccttgcatatctcccttcctgtcactggctttgatcctccttcgttcagcttgtaatcacatcaacagaccaaagacatctctgtgttctgtcaggagagttcacagagccaccaaccctccagaccctgctggttgccgcataaagactctgaggaagggtttgaggctgctgtgatcatgcaatgaatgcatgattgtaccactgcactccagcctgggggataaaggtagatcctgtctaggagagagagagagagaaagagaaagagagagagaagggagggagagacaaagaaaaagagagagagggagggagaaagaaagagagaaagaaaagagaaaagaaagaaaaagaaagaaagagagagagggagggagggagagagaaagaaagaaagaaagagaaagagagaaagagagaaagagaaagaaaggaagaaagaaagaaagaaagaaagaaagaaagaaagaaagaaagaaaagaaaagaaagaaagagagagagaaagaaaagaaagaggaaggaaggaaggaaggaagaaagacaggctctgaggaaggtggcagttcctacaacgggagaaccagtggttaatttgcaaagtggatcctgtggaggcanncagaggagtcccctaggccacccagacagggcttttagctatctgcaggccagacaccaaatttcaggagggctcagtgttaggaatggattatggcttatcaaattcacaggaaactaacatgttgaacagcttttagatttcctgtggaaaatataacttactaaagatggagttcttgtgactgactcctgatatcaagatactgggagccaaattaaaaatcagaaggctgcttggagagcaagtccatgaaatgctctttttcccacagtagaacctatttccctcgtgtctcaaatacttgcacagaggctcactcccttggataatgcagagcgagcacgatacctggcacatactaatttgaataaaatgctgtcaaattcccattcacccattcaagcagcaaactctatctcacctgaatgtacatgccaggcactgtgctagacttggctcaaaaagatttcagtttcctggaggaaccaggagggcaaggtttcaactcagtgctataagaagtgttacaggctggacacggtggctcacgcctgtaatcccaacatttgggaggccgaggcgggcagatcacaaggtcaggagatcgagaccatcctggctaacatggtgaaaccctgtctctactaaaaatacaaaaaattagccgggcgttggcggcaggtgcctgtagtcccagctgctggggaggctgaggcaggagaatggtgtgaacccgggaggcggaacttgcagggggccgagatcgtgccactgcactccagcctgggcgacagagtgagactctgtctcaaaaaaaaaaaaaaagtgttatgatgcagacctgtcaaagaggcaaaggagggtgttcctacactccaggcactgttcataacctggactctcattcattctacaaatggagggctcccctgggcagatccctggagcaggcactttgctggtgtctcggttaaagagaaactgataactcttggtattaccaagagatagagtctcagatggatattcttacagaaacaatattcccacttttcagagttcaccaaaaaatcattttaggcagagctcatctggcattgatctggttcatccatgagattggctagggtaacagcacctggtcttgcagggttgtgtgagcttatctccagggttgccccaactccgtcaggagcctgaaccctgcataccgtatgttctctgccccagccaagaaaggtcaattttctcctcagaggctcctgcaattgacagagagctcccgaggcagagaacagcacccaaggtagagacccacaccctcaatacagacagggagggctattggcccttcattgtacccatttatccatctgtaagtgggaagattcctaaacttaagtacaaagaagtgaatgaagaaaagtatgtgcatgtataaatctgtgtgtcttccactttgtcccacatatactaaatttaaacattcttctaacgtgggaaaatccagtattttaatgtggacatcaactgcacaacgattgtcaggaaaacaatgcatatttgcatggtgatacatttgcaaaatgtgtcatagtttgctactccttgcccttccatgaaccagagaattatctcagtttattagtcccctcccctaagaagcttccaccaatactcttttcccctttcctttaacttgattgtgaaatcaggtattcaacagagaaatttctcagcctcctacttctgcttttgaaagctataaaaacagcgagggagaaactggcagataccaaacctcttcgaggcacaaggcacaacaggctgctctgggattctcttcagccaatcttcattgctcaagtatgactttaatcttccttacaactaggtgctaagggagtctctctgtctctctgcctctttgtgtgtatgcatattctctctctctctctctttctttctctgtctctcctctccttcctctctgcctcctctctcagctttttgcaaaaatgccaggtgtaatataatgcttatgactcgggaaatattctgggaatggatactgcttatctaacagctgacaccctaaaggttagtgtcaaagcctctgctccagctctcctagccaatacattgctagttggggtttggtttagcaaatgcttttctctagacccaaaggacttctctttcacacattcattcatttactcagagatcatttctttgcatgactgccatgcactggatgctgagagaaatcacacatgaacgtagccgtcatggggaagtcactcattttctcctttttacacaggtgtctgaagcagccatggcagaagtacctgagctcgccagtgaaatgatggcttattacaggtcagtggagacgctgagaccagtaacatgagcaggtctcctctttcaagagtagagtgttatctgtgcttggagaccagatttttcccctaaattgcctctttcagtggcaaacagggtgccaagtaaatctgatttaaagactactttcccattacaagtccctccagccttgggacctggaggctatccagatgtgttgttgcaagggcttcctgcagaggcaaatggggagaaaagattccaagcccacaatacaaggaatccctttgcaaagtgtggcttggagggagagggagagctcagattttagctgactctgctgggctagaggttaggcctcaagatccaacagggagcaccagggtgcccacctgccaggcctagaatctgccttctggactgttctgcgcatatcactgtgaaacttgccaggtgtttcaggcagctttgagaggcaggctgtttgcagtttcttatgaacagtcaagtcttgtacacagggaaggaaaaataaacctgtttagaagacataattgagacatgtccctgtttttattacagtggcaatgaggatgacttgttctttgaagctgatggccctaaacagatgaaggtaagactatgggtttaactcccaacccaaggaagggctctaacacagggaaagctcaaagaagggagttctgggccactttgatgccatggtattttgttttagaaagactttaacctcttccagtgagacacaggctgcaccacttgctgacctggccacttggtcatcatatcaccacagtcactcactaacgttggtggtggtggccacacttggtggtgacaggggaggagtagtgataatgttcccatttcatagtaggaagacaaccaagtcttcaacataaatttgattatccttttaagagatggattcagcctatgccaatcacttgagttaaactctgaaaccaagagatgatcttgagaactaacatatgtctaccccttttgagtagaatagttttttgctacctggggtgaagcttataacaacaagacatagatgatataaacaaaaagatgaattgagacttgaaagaaaaccattcacttgctgtttgaccttgacaagtcattttacccgctttggacctcatctgaaaaataaagggctgagctggatgatctctgagattccagcatcctgcaacctccagttctgaaatattttcagttgtagctaagggcatttgggcagcaaatggtcatttttcagactcatccttacaaagagccatgttatattcctgctgtcccttctgttttatatgatgctcagtagccttcctaggtgcccagccatcagcctagctaggtcagttgtgcaggttggaggcagccacttttctctggctttattttattccagtttgtgatagcctcccctagcctcataatccagtcctcaatcttgttaaaaacatatttctttagaagttttaagactggcataacttcttggctgcagctgtgggaggagcccattggcttgtctgcctggcctttgccccccattgcctcttccagcagcttggctctgctccaggcaggaaattctctcctgctcaactttcttttgtgcacttacaggtctctttaactgtctttcaagcctttgaaccattatcagccttaaggcaacctcagtgaagccttaatacggagcttctctgaataagaggaaagtggtaacatttcacaaaaagtactctcacaggatttgcagaatgcctatgagacagtgttatgaaaaaggaaaaaaaagaacagtgtagaaaaattgaatacttgctgagtgagcataggtgaatggaaaatgttatggtcatctgcatgaaaaagcaaatcatagtgtgacagcattagggatacaaaaagatatagagaaggtatacatgtatggtgtaggtggggcatgtacaaaaagatgacaagtagaatcgggatttattctaaagaatagcctgtaaggtgtccagaagccacattctagtcttgagtctgcctctacctgctgtgtgcccttgagtacacccttaacctccttgagcttcagagagggataatctttttattttattttattttattttgttttgttttgttttgttttgttttatgagacagagtctcactctgttgcccaggctggagtgcagtggtacaatcttggcttactgcatcctccacctcctgagttcaagcgattctccttcctcagtctcctgaatagctaggattacaggtgcaccccaccacacccagctaatttttgtatttttagtagagaaggggtttcgccatgttggccaggctggttttgaagtcctgacctaaatgattcatccacctcggcttcccaaagtgctgggattacaggcatgagccaccacgcctggcccagagagggatgatctttagaagctcgggattctttcaagccctttcctcctctctgagctttctactctctgatgtcaaagcatggttcctggcaggaccacctcaccaggctccctccctcgctctctccgcagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaaggtagttagccaagagcaggcagtagatctccacttgtgtcctcttggaagtcatcaagccccagccaactcaattcccccagagccaaagccctttaaaggtagaaggcccagcggggagacaaaacaaagaaggctggaaaccaaagcaatcatctctttagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccacctcgcctcacgaggcctgcccttctgattttatacctaaacaacatgtgctccacatttcagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaacatcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcactggccagtgcatttgttaacaggagtgtgaccctgagaaattagacggctcaagcactcccaggaccatgtccacccaagtctcttgggcatagtgcagtgtcaattcttccacaatatggggtcatttgatggacatggcctaactgcctgtgggttctctcttcctgttgttgaggctgaaacaagagtgctggagcgataatgtgtccatccccctccccagtcttccccccttgccccaacatccgtcccacccaatgccaggtggttccttgtagggaaattttaccgcccagcaggaacttatatctctccgctgtaacgggcaaaagtttcaagtgcggtgaacccatcattagctgtggtgatctgcctggcatcgtgccacagtagccaaagcctctgcacaggagtgtgggcaactaaggctgctgactttgaaggacagcctcactcagggggaagctatttgctctcagccaggccaagaaaatcctgtttctttggaatcgggtagtaagagtgatcccagggcctccaattgacactgctgtgactgaggaagatcaaaatgagtgtctctctttggagccactttcccagctcagcctctcctctcccagtttcttcccatgggctactctctgttcctgaaacagttctggtgcctgatttctggcagaagtacagcttcacctctttcctttccttccacattgatcaagttgttccgctcctgtggatgggcacattgccagccagtgacacaatggcttccttccttccttccttcagcatttaaaatgtagaccctctttcattctccgttcctactgctatgaggctctgagaaaccctcaggcctttgaggggaaaccctaaatcaacaaaatgaccctgctattgtctgtgagaagtcaagttatcctgtgtcttaggccaaggaacctcactgtgggttcccacagaggctaccaattacatgtatcctactctcggggctaggggttggggtgaccctgcatgctgtgtccctaaccacaagacccccttctttcttcagtggtgttctccatgtcctttgtacaaggagaagaaagtaatgacaaaatacctgtggccttgggcctcaaggaaaagaatctgtacctgtcctgcgtgttgaaagatgataagcccactctacagctggaggtaagtgaatgctatggaatgaagcccttctcagcctcctgctaccacttattcccagacaattcaccttctccccgcccccatccctaggaaaagctgggaacaggtctatttgacaagttttgcattaatgtaaataaatttaacataatttttaactgcgtgcaaccttcaatcctgctgcagaaaattaaatcattttgccgatgttattatgtcctaccatagttacaaccccaacagattatatattgttagggctgctctcatttgatagacaccttgggaaatagatgacttaaagggtcccattatcacgtccactccactcccaaaatcaccaccactatcacctccagctttctcagcaaaagcttcatttccaagttgatgtcattctaggaccataaggaaaaatacaataaaaagcccctggaaactaggtacttcaagaagctctagcttaattttcacccccccaaaaaaaaaaaattctcacctacattatgctcctcagcatttggcactaagttttagaaaagaagaagggctcttttaataatcacacagaaagttgggggcccagttacaactcaggagtctggctcctgatcatgtgacctgctcgtcagtttcctttctggccaacccaaagaacatctttcccataggcatctttgtcccttgccccacaaaaattcttctttctctttcgctgcagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttgattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaagaaaaaaaaaaaagggtctctcctgatcattgactgtctggattgacactgacagtaagcaaacaggctgtgagagttcttgggactaagcccactcctcattgctgagtgctgcaagtacctagaaatatccttggccaccgaagactatcctcctcacccatcccctttatttcgttgttcaacagaaggatattcagtgcacatctggaacaggatcagctgaagcactgcagggagtcaggactggtagtaacagctaccatgatttatctatcaatgcaccaaacatctgttgagcaagcgctatgtactaggagctgggagtacagagatgagaacagtcacaagtccctcctcagataggagaggcagctagttataagcagaacaaggtaacatgacaagtagagtaagatagaagaacgaagaggagtagccaggaaggagggaggagaacgacataagaatcaagcctaaagggataaacagaagatttccacacatgggctgggccaattgggtgtcggttacgcctgtaatcccagcactttgggtggcaggggcagaaagatcgcttgagcccaggagttcaagaccagcctgggcaacatagtgagactcccatctctacaaaaaataaataaataaataaaacaatcagccaggcatgctggcatgcacctgtagtcctagctacttgggaagctgacactggaggattgcttgagcccagaagttcaagactgcagtgagcttatccgttgacctgcaggtcgac

SEQ ID NO: 42 - DNA, 13 bp, Artificial Sequence
  Description of Artificial Sequence Synthetic construct sequence
ttgcatagggtct

SEQ ID NO: 43 - DNA, 12 bp, Artificial Sequence
  Description of Artificial Sequence Synthetic construct sequence
ttgcatagggtc

SEQ ID NO: 44 - DNA, 12 bp, Artificial Sequence
  Description of Artificial Sequence Synthetic construct sequence
tgcatagggtct

SEQ ID NO: 45 - DNA, 11 bp, Artificial Sequence
  Description of Artificial Sequence Synthetic construct sequence
tgcatagggtc

SEQ ID NO: 46 - DNA, 12 bp, Artificial Sequence
  Description of Artificial Sequence Synthetic construct sequence
ttgcatagggtc

SEQ ID NO: 47 - DNA, 12 bp, Artificial Sequence
  Description of Artificial Sequence Synthetic construct sequence
ctgcatagggtc

SEQ ID NO: 48 - DNA, 11 bp, Artificial Sequence
  Description of Artificial Sequence Synthetic construct sequence
tgcatagggtc

SEQ ID NO: 49 - DNA, 11 bp, Artificial Sequence
  Description of Artificial Sequence Synthetic construct sequence
cgcatagggtc

SEQ ID NO: 50 - DNA, 11 bp, Artificial Sequence
  Description of Artificial Sequence Synthetic construct sequence
tacatagggtc

SEQ ID NO: 51 - DNA, 11 bp, Artificial Sequence
  Description of Artificial Sequence Synthetic construct sequence
tgtatagggtc

SEQ ID NO: 52 - DNA, 11 bp, Artificial Sequence
  Description of Artificial Sequence Synthetic construct sequence
tgcgtagggtc

SEQ ID NO: 53 - DNA, 11 bp, Artificial Sequence
  Description of Artificial Sequence Synthetic construct sequence
tgcacagggtc

SEQ ID NO: 54 - DNA, 11 bp, Artificial Sequence
  Description of Artificial Sequence Synthetic construct sequence
tgcatggggtc

SEQ ID NO: 55 - DNA, 11 bp, Artificial Sequence
  Description of Artificial Sequence Synthetic construct sequence
tgcataaggtc

SEQ ID NO: 56 - DNA, 11 bp, Artificial Sequence
  Description of Artificial Sequence Synthetic construct sequence
tgcatagagtc

SEQ ID NO: 57 - DNA, 11 bp, Artificial Sequence
  Description of Artificial Sequence Synthetic construct sequence
tgcataggctc

SEQ ID NO: 58 - DNA, 11 bp, Artificial Sequence
  Description of Artificial Sequence Synthetic construct sequence
tgcatagggcc

SEQ ID NO: 59 - DNA, 11 bp, Artificial Sequence
  Description of Artificial Sequence Synthetic construct sequence
tgcatagggtt

SEQ ID NO: 60 - DNA, 12 bp, Artificial Sequence
  Description of Artificial Sequence Synthetic construct sequence
tgcatagggtct

SEQ ID NO: 61 - DNA, 12 bp, Artificial Sequence
  Description of Artificial Sequence Synthetic construct sequence
tgcatagggtcc

SEQ ID NO: 62 - DNA, 12 bp, Artificial Sequence
  Description of Artificial Sequence Synthetic construct sequence
ttgcatagggtc

SEQ ID NO: 63 - DNA, 12 bp, Artificial Sequence
  Description of Artificial Sequence Synthetic construct sequence
ctgcatagggtc

SEQ ID NO: 64 - DNA, 11 bp, Artificial Sequence
  Description of Artificial Sequence Synthetic construct sequence
tgcatagggtc

SEQ ID NO: 65 - DNA, 11 bp, Artificial Sequence
  Description of Artificial Sequence Synthetic construct sequence
cgcatagggtc

SEQ ID NO: 66 - DNA, 11 bp, Artificial Sequence
  Description of Artificial Sequence Synthetic construct sequence
tacatagggtc

SEQ ID NO: 67 - DNA, 11 bp, Artificial Sequence
  Description of Artificial Sequence Synthetic construct sequence
tgtatagggtc

SEQ ID NO: 68 - DNA, 11 bp, Artificial Sequence
  Description of Artificial Sequence Synthetic construct sequence
tgcgtagggtc

SEQ ID NO: 69 - DNA, 11 bp, Artificial Sequence
  Description of Artificial Sequence Synthetic construct sequence
tgcacagggtc

SEQ ID NO: 70 - DNA, 11 bp, Artificial Sequence
  Description of Artificial Sequence Synthetic construct sequence
tgcatggggtc

SEQ ID NO: 71 - DNA, 11 bp, Artificial Sequence
  Description of Artificial Sequence Synthetic construct sequence
tgcataaggtc

SEQ ID NO: 72 - DNA, 11 bp, Artificial Sequence
  Description of Artificial Sequence Synthetic construct sequence
tgcatagagtc

SEQ ID NO: 73 - DNA, 11 bp, Artificial Sequence
  Description of Artificial Sequence Synthetic construct sequence
tgcataggcct

SEQ ID NO: 74 - DNA, 11 bp, Artificial Sequence
  Description of Artificial Sequence Synthetic construct sequence
tgcatagggtt

SEQ ID NO: 75 - DNA, 11 bp, Artificial Sequence
  Description of Artificial Sequence Synthetic construct sequence
tgcatagggcc

SEQ ID NO: 76 - DNA, 12 bp, Artificial Sequence
  Description of Artificial Sequence Synthetic construct sequence
tgcatagggctc

SEQ ID NO: 77 - DNA, 12 bp, Artificial Sequence
  Description of Artificial Sequence Synthetic construct sequence
tgcatagggctt

SEQ ID NO: 78 - DNA, 12 bp, Artificial Sequence
  Description of Artificial Sequence Synthetic consensus sequence
ggggrnyyyccc